(19) United States
(12) Patent Application Publication
(10) Pub. No.: US 2018/0201994 A1
(43) Pub. Date: Jul. 19, 2018

(54) BALANCED CAPTURE PROBES AND METHODS OF USE THEREOF

(71) Applicant: Counsyl, Inc., South San Francisco, CA (US)

(72) Inventors: Kyle BEAUCHAMP, San Francisco, CA (US); Dale MUZZEY, San Francisco, CA (US); Noah WELKER, Half Moon Bay, CA (US); Jared Robert MAGUIRE, Oakland, CA (US)

(21) Appl. No.: 15/873,648

(22) Filed: Jan. 17, 2018

Related U.S. Application Data
(60) Provisional application No. 62/447,816, filed on Jan. 18, 2017.
(60) Provisional application No. 62/487,879, filed on Apr. 20, 2017.

(51) Int. Cl. C12Q 1/6869; C12N 15/10; C12Q 1/6806; C12Q 1/6883; C40B 50/14; C12Q 1/6811; G06F 19/22; B01J 19/00

(52) U.S. Cl. CPC C12Q 1/6869; C12N 15/1093; C12Q 1/6806; B01J 19/0046; C40B 50/14; C12Q 1/6811; G06F 19/22; C12Q 1/6883

(57) ABSTRACT

Capture probe libraries can be used to enrich a region of interest in a sequencing library for high-depth sequencing. The capture probes within the capture probe libraries often do not function in a predictable or uniform manner. Described herein are balanced capture probe libraries and methods of balancing capture probe libraries. A sequencing library can be enriched using balanced capture probe libraries, and the enriched sequencing library can be sequenced to obtain a sequencing depth closer to a desired sequencing depth.

### CROSS-REFERENCE TO RELATED APPLICATION

This application claims priority benefit of U.S. Provisional Application No. 62/447,816, filed on Jan. 18, 2017, entitled “BALANCED CAPTURE PROBES AND METHODS OF USE THEREOF”; and U.S. Provisional Application No. 62/487,879, filed on Apr. 20, 2017, entitled “BALANCED CAPTURE PROBES AND METHODS OF USE THEREOF”; each of which is incorporated herein by reference for all purposes.

### TECHNICAL FIELD

The present invention relates to capture probe libraries useful for enriching sequencing libraries and methods of preparing the same.

### BACKGROUND

Massive parallel sequencing (also referred to as next-generation sequencing (NGS)) is a high-throughput process for sequencing nucleic acids. The process includes generating sequencing reads, often in the order of millions sequencing reads, from a sample nucleic acid library. Deep sequencing all regions of a genome with sufficient resolution for accurate identification of genetic variants (such as SNPs, indels, or copy number variants) is generally impractical when interrogating a large number of samples due to the cost and time required to sequence. Therefore, it is preferable to focus sequencing efforts on specific regions of interest when sequencing a large number of samples. For example, a patient may only be interested in sequencing information relating to a small portion of the genome, such as 100 to 200 genes that are related to more common genetic diseases.

Capture probes can be used to enrich a sequencing library for particular regions of interest. Sequencing libraries contain nucleic acid molecules (such as fragmented genomic DNA or cell-free DNA fragments) from regions throughout the genome. Capture probes are generally DNA or RNA oligonucleotides that hybridize to nucleic acid molecules in the sequencing library with complementary segments. The capture probes can be selected based on the region of interest such that those nucleic acid molecules in the sequencing library containing a portion of the region of interest hybridize to the capture probes and can be enriched, whereas those nucleic acid molecules in the sequencing library that do not contain a portion of the region of interest do not hybridize to the capture probes and are not enriched.

Enrichment of sequencing libraries using capture probes allows for more efficient high-throughput sequencing of regions of interest. This efficiency keeps the overall cost of sequencing samples down while maintaining or increasing the sensitivity and specificity of a diagnostic test or screen.

The disclosures of all publications, patents, and patent applications referred to herein are each hereby incorporated by reference in their entireties. To the extent that any reference incorporated by reference conflicts with the instant disclosure, the instant disclosure shall control.

### SUMMARY

Described herein is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; selecting a subsequent known amount of each capture probe based on the initial known amount and the sequencing depth attributable to said capture probe, wherein the subsequent known amount of each capture probe is selected to minimize a difference between a predicted sequencing depth profile and a desired sequencing depth profile, and wherein minimization of the difference is constrained by a minimum subsequent amount or a maximum subsequent amount of each capture probe; and constructing the balanced capture probe library by combining at least a fraction of the capture probes at the subsequent known amount of each capture probe in the balanced capture probe library. In some embodiments, the desired sequencing depth profile is non-uniform.

Also described herein is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; selecting a subsequent known amount of each capture probe based on the initial known amount and the sequencing depth attributable to said capture probe, wherein the subsequent known amount of each capture probe is selected to minimize a difference between a predicted sequencing depth profile and a non-uniform desired sequencing depth profile; and constructing the balanced capture probe library by combining at least a fraction of the capture probes at the subsequent known amount for each capture probe in the balanced capture probe library.

In some embodiments, the difference between the predicted sequencing depth profile and the desired sequencing depth profile is defined by an objective function. In some embodiments, the desired sequencing depth profile is implied by the objective function.

In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount.

In some embodiments, the sequencing depth is based on a number of sequencing reads that comprise a sequence substantially complementary to a sequence within the capture probe. In some embodiments, the sequencing depth is based on a number of sequencing reads that comprise a sequence substantially complementary to at least half of the capture probe. In some embodiments, the number of sequencing reads is a number of consensus sequencing reads. In some embodiments, the number of sequencing reads is a number of duplex consensuses sequencing reads.

In some embodiments, the capture probes are not substantially complementary to overlapping portions of the region of interest. In some embodiments, at least two capture probes in the plurality of capture probes are substantially complementary to overlapping portions of the region of interest.

In some embodiments, the method comprises obtaining for each capture probe a binding fraction based on the sequencing depth attributable to the capture probe; and wherein the subsequent known amount is based on the initial known amount and the binding fraction. In some embodiments, the method comprises obtaining for each capture probe a binding fraction, wherein the binding fraction is determined for the fraction of nucleic acid molecules comprising a segment substantially complementary to at least half of the capture probe that bound to the capture probe during enrichment of the sequencing library based on the sequencing depth attributable to the capture probe; and wherein the subsequent known amount is based on the initial known amount and the fraction. In some embodiments, obtaining the binding fraction comprises approximating the sequencing depth for a given capture probe i as a Poisson distribution:

dᵢₛ=Poisson(Nₛπᵢ)

wherein dᵢₛ is the determined sequencing depth attributable to the capture probe i; Nₛ is the number of nucleic acid molecules in sequencing library s; and πᵢ is the binding fraction for capture probe i. In some embodiments, πᵢ is determined by fitting a maximum likelihood model or a Markov chain Monte Carlo model.

In some embodiments, the difference is defined by an objective function of the subsequent amounts of the capture probes in the balanced capture probe library, according to:

$$f ( \vec{v} ) = V ( \vec{π} ( \vec{v} ) )$$

$$wherein : {( \vec{π} ( \vec{v} ) )}_{i} = {( 1 + \frac{{γ}_{i}}{{v}_{i} {c}_{i}} )}^{- 1}$$

Wherein V( ) is a user-defined objective function for the capture probe library; (\vec{π}(\vec{v}))ᵢ is the binding fraction for capture probe i for the vector \vec{π}(\vec{v}); cᵢ is an effective concentration for capture probe i for the vector \vec{π}(\vec{v}); vᵢ is the subsequent amount of capture probe i for the vector \vec{π}(\vec{v}); and γᵢ is the initial amount of capture probe i for the vector \vec{π}(\vec{v}).

In some embodiments, the difference is an objective function defined by a coefficient of variation.

In some embodiments, the plurality of capture probes comprises 10 or more unique capture probes. In some embodiments, the capture probes are about 20 to about 160 bases in length. In some embodiments, the plurality of capture probes comprise DNA capture probes. In some embodiments, the plurality of capture probes comprise RNA capture probes. In some embodiments, the capture probes are biotinylated.

In some embodiments, the method of preparing a balanced capture probe library further comprises enriching the sequencing library using the balanced capture probe library; sequencing the sequencing library enriched using the balanced capture probe library; determining a sequencing depth attributable to each capture probe in the balanced capture probe library; selecting a second subsequent known amount of each capture probe based on the subsequent known amount of said capture probe in the balanced capture probe library and the sequencing depth attributable to said capture probe, wherein the second subsequent known amount of each capture probe is selected to minimize a difference between a second predicted sequencing depth profile and the desired sequencing depth profile; and constructing a re-balanced capture probe library by combining at least a fraction of the capture probes at the second subsequent known amount of each capture probe in the re-balanced capture probe library. In some embodiments, the difference is constrained by a minimum second subsequent amount or a maximum second subsequent amount of each capture probe.

In some embodiments of the method of preparing a balanced capture probe library, constructing the balanced capture probe library comprises combining each capture probe in the first capture probe library at the subsequent known amount of each capture probe in the balanced capture probe library.

In some embodiments, the sequencing library is an RNA or a DNA sequencing library.

Further provided herein is a balanced capture probe library made according to any one of the methods described above.

Also provided herein is a method of enriching a test sequencing library comprising combining the test sequencing library comprising nucleic acid molecules with the capture probe library prepared according to any one of the methods described above; and selecting nucleic acid molecules from the test sequencing library that hybridize with the capture probes in the capture probe library. In some embodiments, the method further comprises amplifying the enriched test sequencing library. In some embodiments, the method further comprises removing the capture probes from the enriched test sequencing library.

Additionally, there is provided a method of sequencing a test sequencing library comprising enriching the test sequencing library according to the method described above; and sequencing the enriched test sequencing library. In some embodiments, the method further comprises amplifying the enriched test sequencing library. In some embodiments, the method further comprises removing the capture probes from the enriched test sequencing library.

In some embodiments, the test sequencing library comprises cell-free DNA. In some embodiments, the cell-free DNA comprises fetal cell-free DNA. In some embodiments, the cell-free DNA comprises circulating tumor cell-free DNA. In some embodiments, the test sequencing library comprises fragmented DNA derived from cells contained with a sample. In some embodiments, the test sequencing library is an RNA sequencing library. In some embodiments, the nucleic acid molecules in the test sequencing library have an average length of about 100 bases to about 500 bases. In some embodiments, the nucleic acid molecules in the test sequencing library are ligated to sequencing adapters comprising molecular barcodes.

In some embodiments of the methods described above, enriching the sequencing library comprises separating nucleic acid molecules in the sequencing library that are hybridized to the capture probes from nucleic acid molecules in the sequencing library that are not hybridized to the capture probes.

In some embodiments, there is a method of forming a pooled capture probe library comprising combing two or more balanced capture probe libraries prepared according to any of the methods described above.

In another aspect, there is provided a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the one or more contiguous loci within the tile; and constructing the balanced capture probe library by combining at least a fraction of the capture probes at the subsequent known amount of each capture probe in the balanced capture probe library. In some embodiments, the desired sequencing depth profile is a predetermined minimum sequencing depth at each of the one or more contiguous loci. In some embodiments, the subsequent amount is further selected to obtain a sequencing depth below a maximum sequencing depth. In some embodiments, the tile comprises a plurality of loci and the desired sequencing depth profile is uniform for the plurality of loci within the tile. In some embodiments, the tile comprises a plurality of loci and the desired sequencing depth profile is non-uniform for the plurality of loci within the tile.

In some embodiments, the method of preparing a balanced capture probe library further comprises enriching the sequencing library using the first capture probe library by extending the capture probes that hybridize to the nucleic acid molecules.

In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles.

In some embodiments, the desired sequencing depth profile is uniform for each of the tiles in the plurality of tiles. In some embodiments, the desired sequencing depth profile is non-uniform for each of the tiles in the plurality of tiles.

In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount.

In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands.

In some embodiments, selecting the subsequent known amount of each capture probe comprises simulating a sequencing depth for at least one simulated capture probe amount. In some embodiments, selecting the subsequent known amount of each capture probe comprises combining a simulated sequencing depth determined for two or more capture probes. In some embodiments, the simulated sequencing depth is simulated using a proportional relationship between the amount of the capture probe and the sequencing depth at the locus.

In some embodiments of the method of preparing a balanced capture probe library, the method comprises sequencing a plurality of sequencing libraries; and the sequencing depth at each locus is a minimum confidence sequencing depth. In some embodiments, the minimum confidence sequencing depth is a median sequencing depth minus two times an interquartile range determined from a distribution of sequencing depths at each locus from the plurality of sequencing libraries.

In some embodiments, constructing the balanced capture probe library by combining at least a fraction of the capture probes comprises omitting (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, the method of preparing a balanced capture probe library further comprises enriching the sequencing library using the balanced capture probe library; sequencing the sequencing library enriched using the balanced capture probe library; determining at each locus within the tile: (i) a sequencing depth attributable to each capture probe in the balanced capture probe library, and (ii) a sequencing depth attributable to the balanced capture probe library; selecting a second subsequent known amount of each capture probe based on the subsequent known amount of said capture probe, the sequencing depth at each locus attributable to said capture probe, and the sequencing depth at each locus attributable to the balanced capture probe library, wherein the second subsequent amount is selected to obtain a desired sequencing depth profile at substantially all of the one or more contiguous loci within the tile; and constructing a re-balanced capture probe library by combining at least a fraction of the capture probes at the second subsequent known amount of each capture probe in the re-balanced capture probe library.

In some embodiments, the capture probes are about 20 to about 60 bases in length.

In some embodiments, there is provided a balanced capture probe library made according to any one of the methods described above.

In some embodiments, there is provided a method of enriching a test sequencing library comprising combining the test sequencing library comprising test nucleic acid molecules with the capture probe library prepared according to any one of the methods described above; and extending the capture probes that hybridize to the test nucleic acid molecules. In some embodiments, the method further comprises amplifying the enriched test sequencing library.

In some embodiments, there is provided a method of sequencing a test sequencing library comprising enriching the test sequencing library according to the method described above, and sequencing the enriched test sequencing library. In some embodiments, the method further comprises amplifying the enriched test sequencing library.

In some embodiments, the test sequencing library comprises fragmented DNA derived from cells in a sample. In some embodiments, the test sequencing library comprises cell-free DNA.

In some embodiments, there is provided a method of forming a pooled capture probe library comprising combining two or more balanced capture probe libraries made according to any one of the methods described above.

### BRIEF DESCRIPTION OF THE DRAWINGS

FIG. 1 illustrates a schematic of sequencing depth attributable to several capture probes which are used to enrich a region of interest by extending the capture probe and using a plurality of contiguous loci as a template for the extension. The sequencing depth at each of the contiguous loci attributable to each capture probe can be determined, and the sequencing depths attributable to each capture probe at each locus can be summed to determine the sequencing depth attributable to the capture probe library at each locus.

FIGS. 2A-I each represent a sequencing depth profile of a single probe used to enrich 96 different samples. These profiles include FIG. 2A, which include sequencing depth profiles with exponential decay; FIG. 2B, which shows sequencing depth profiles that are highly variable from sample to sample, and have many increases and decreases in the sequencing depth as a function of distance from the capture probe, although with a general trend toward decreased sequencing depth; FIG. 2C, which include flat sequencing depth profiles and linear decay of sequencing depth; FIG. 2D, which include sequencing depth profiles with a humped pattern; FIG. 2E, which include plateaued sequencing depth profiles with an even sequencing depth for a first distance from the capture probe (to about 200 bases in the illustrated example) before a beginning a decay in sequencing depth; FIG. 2F, which include sequencing depth profiles that exhibit rapid decay of the sequencing depth in a short distance; FIG. 2G, which include sequencing depth profiles that have low sequencing depth close to the capture probe and decay with distance from the capture probe; FIG. 2H which include sequencing depth profiles with no or essentially no sequencing depth regardless of distance from the capture probe; and FIG. 2I, which include noise sequencing depth profiles that exhibit rapid decay of the sequencing depth in a short distance.

FIG. 3A illustrates sequencing depth of the MSH2 gene for 96 samples using more than 100 different capture probes in the capture probe library which were used to enrich the MSH2 gene by extending the capture probes using the MSH2 gene as a template for the extension. The bold line represents the minimum number of sequencing reads obtained across any of the 96 samples. The shaded box indicates a portion of the gene with a sequencing depth lower than the predetermined minimum sequencing depth (20-fold coverage).

FIG. 3B illustrates sequencing depth across the >3500 tiles covering portions of >200 different genes using >12000 different capture probes. The capture probes were used to enrich the genes by extending the capture probe using contiguous loci within the tiles as a template for the extension.

FIG. 4 illustrates an exemplary process for simulating capture probe amounts and sequencing depths based on determined sequencing depths at known capture probe amounts. In the illustrated example, the capture probe amounts are simulated for three iterations with progressively narrow windows of capture probe amounts.

FIG. 5A shows a histogram plotting the number of capture probes in the capture probe library for which a given number of collapsed sequencing reads with at least 50% overlap with a probe can be attributed prior to balancing the capture probe library. FIG. 5B shows a histogram plotting the number of capture probes in the capture probe library for which a given number of collapsed sequencing reads with at least 50% overlap with a probe can be attributed after to balancing the capture probe library. The capture probes enriched the region of interest by hybridizing to nucleic acid molecules in the sequencing library and isolating the hybridized nucleic acid molecules.

FIG. 6A shows a histogram plotting the number of individual bases in the region of interest that obtained a given number of collapsed sequencing reads prior to balancing the capture probe library. FIG. 6B s shows a histogram plotting the number of individual bases in the region of interest that obtained a given number of collapsed sequencing reads after balancing the capture probe library. The capture probes enriched the region of interest by hybridizing to nucleic acid molecules in the sequencing library and isolating the hybridized nucleic acid molecules.

FIG. 7A shows the log₁₀ sequencing depth attributable to the second percentile of each capture probe (that is, the sequencing depth reported is the second lowest sequencing depth for each capture probe among the 96 samples) in the capture probe library plotted against the position within the region of interest prior to balancing the capture probe library. FIG. 7B shows the log₁₀ sequencing depth attributable to the second percentile of each capture probe in the capture probe library plotted against the position within the region of interest after balancing the capture probe library. The capture probe library enriched the region of interest by extending the capture probe using contiguous loci within tiles of the region of interest as a template.

FIG. 8 illustrates a method of enriching a nucleic acid library using a direct targeted sequencing method in solution.

### DETAILED DESCRIPTION

Provided herein there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; selecting a subsequent known amount of each capture probe based on the initial known amount and the sequencing depth attributable to said capture probe, wherein the subsequent known amount of each capture probe is selected to minimize a difference between a predicted sequencing depth profile and a desired sequencing depth profile; and constructing the balanced capture probe library by combining at least a fraction of the capture probes at the subsequent known amount for each capture probe in the balanced capture probe library. The difference can be defined by an objective function, such as a coefficient of variation. The desired sequencing library can be implied by the objective function. In some embodiments, the desired sequencing depth profile is non-uniform. In some embodiments, minimization of the difference is constrained by a minimum subsequent amount or a maximum subsequent amount of each capture probe. The initial known amount can be, for example, an initial known volume, and the subsequent known amount can be a subsequent known volume. In some embodiments, the initial known amount is an initial known mass or an initial known number of moles, and the subsequent known amount is a subsequent known mass or a subsequent known number of moles. The process can be iteratively repeated to further balance the capture probe library, as desired. In some embodiments, two or more balanced capture probe libraries are pooled to form a pooled capture probe library.

Further provided herein is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining at each locus within a tile of the region of interest comprising one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the contiguous loci within the tile; and constructing the balanced capture probe library by combining at least a fraction of the capture probes at the subsequent known amount of each capture probe in the balanced capture probe library. In some embodiments, the subsequent amount is further selected to obtain a sequencing depth below a maximum sequencing depth. The sequencing library can be enriched, for example, by extending the capture probes that hybridize to the nucleic acid molecules. The initial known amount can be, for example, an initial known volume, and the subsequent known amount can be a subsequent known volume. In some embodiments, the initial known amount is an initial known mass or an initial known number of moles, and the subsequent known amount is a subsequent known mass or a subsequent known number of moles. The process can be iteratively repeated to further balance the capture probe library, as desired. In some embodiments, two or more balanced capture probe libraries are pooled to form a pooled capture probe library.

Also provided herein there is a method of enriching a test sequencing library comprising combining the test sequencing library comprising test nucleic acid molecules with the balanced capture probe library; and selecting nucleic acid molecules from the test sequencing library that hybridize with the capture probes in the balanced capture probe library.

Further provided herein there is a method of enriching a test sequencing library comprising combining the test sequencing library comprising test nucleic acid molecules with the balanced capture probe library and extending the capture probes that hybridize to the test nucleic acid molecules.

Additionally, there is provided a method of sequencing a test sequencing library comprising enriching the test sequencing library using a balanced capture probe library and sequencing the enriched test sequencing library.

Capture probes in an unbalanced capture probe library generally do not uniformly enrich corresponding portions of a region of interest, as some capture probes are more efficient at enriching corresponding segments of the regions of the genome than other capture probes. For example, variations in GC content in various portions of the genome can result in different enrichment efficiency of the capture probes. Thus, an equimolar amount of unique capture probes in the capture probe library does not necessarily result in a uniform sequencing depth profile.

In some embodiments, it is preferable to obtain a controlled (or desired) sequencing depth profile across a region of interest. In some embodiments, it is preferable to obtain a desired but non-uniform sequencing depth profile. This may be desirable if, for example, the first portion of the region of interest and the second portion of the region of interest have a different minimum sequencing depths necessary to obtain a desired resolution.

In some embodiments, it is preferable to obtain a predetermined minimum sequencing depth throughout the region of interest or a portion of the region of interest. For example, when interrogating a region of interest for a SNP or indel variant, it can be desirable to obtain a predetermined minimum sequencing depth to ensure sufficient resolution to make a variant call. If the region of interest is not sequenced to the desired sequencing depth, the variant call will be made with lower confidence or may not be made at all. Obtainment of a predetermined minimum sequencing depth throughout the region of interest or portion of the region of interest can be, but need not be, combined with obtainment of a predetermined sequencing depth profile throughout the region of interest.

In some embodiments, the amounts of the capture probe in the balanced capture probe library are further selected to obtain a sequencing depth below a maximum sequencing depth, which is set above the predetermined minimum sequencing depth. Obtaining large numbers of sequencing reads can be expensive, particularly when sequencing a large number of samples. While sequencing above a predetermined minimum sequencing depth may be desirable in some applications, increased sequencing depth much beyond the predetermined sequencing depth may offer diminishing returns. Thus, in some embodiments, it is desirable to obtain a sequencing depth below a predetermined maximum sequencing depth. The predetermined maximum sequencing depth can be independent from, or combined with, obtainment of a desired sequencing depth profile.

The methods described herein are useful for preparing balanced capture probe libraries. The balanced capture probe libraries can be prepared to obtain a desired sequencing depth profile (which may be uniform or non-uniform) throughout the region of interest (or at substantially all of a plurality of loci), to obtain a minimum sequencing depth at one or more portions (e.g., contiguous loci) of the region of interest, or to minimize a total number of sequencing reads.

Altering the relative amounts of capture probes in a first (e.g., unbalanced) capture probe library can account for differences in enrichment efficiency of the capture probes in the capture probe library. Thus, the balanced capture probe library can be used to obtain a desired sequencing depth profile. Because changes in the relative amounts of capture probes can have an unexpected effect on other capture probes in the capture probe library, in some embodiments, adjustments are constrained by a minimum or maximum amount used to construct the balanced sequencing library. This has the benefit of limiting extreme changes during the preparation of the balanced capture probe library. Additionally, the amount of capture probe used to construct the balanced sequencing library may be limited by certain volume constraints or availability of the capture probe. By constraining the minimum and maximum amounts of each capture probe used to construct the capture probe library, the undesirable effects of using too much or too little capture probe are minimized. The capture probe library can be iteratively balanced. That is, after preparing a first balanced capture probe library, the balanced capture probe library can be rebalanced. This allows for incremental changes when rebalancing the capture probe library and the determination of optimized relative amounts of the capture probes in the capture probe libraries.

### Definitions

As used herein, the singular forms “a,” “an,” and “the” include the plural reference unless the context clearly dictates otherwise.

Reference to “about” or “approximately” a value or parameter herein includes (and describes) variations that are directed to that value or parameter per se. For example, description referring to “about X” includes description of “X”.

A “portion adjacent to a region of interest” includes a portion adjacent to a sub-region of the region of interest. Reference to a “portion of or adjacent to a region of interest” refers to a sequence that 1) is entirely within the region of interest, 2) is entirely outside but adjacent to the region of interest, or 3) includes a contiguous sequence from within and adjacent to the region of interest. Reference to a “sequence that is substantially complementary to a portion of or adjacent to a region of interest” refers to 1) a sequence that is substantially complementary to a sequence entirely within the region of interest, 2) a sequence substantially complementary to a sequence entirely outside but adjacent to the region of interest, or 3) a sequence that is substantially complementary to a contiguous sequence from with and adjacent to the region of interest.

The term “average” as used herein refers to either a mean or a median, or any value used to approximate the mean or the median, unless the context clearly indicates otherwise.

It is understood that aspects and variations of the invention described herein include “consisting” and/or “consisting essentially of” aspects and variations.

The term “substantially complementary” is used to refer to two nucleic acid sequences (X and Y) on opposite strands for which both are at least 12 bases in length and the complementarity fraction between them is at least 0.75. The complementarity fraction is calculated as follows. First, the optimal alignment between X and the reverse complement of Y is calculated with the Needleman-Wunsch algorithm (Needleman et al., A general method applicable to the search for similarities in the amino acid sequence of two proteins. Journal of Molecular Biology, vol. 48 (3), pp. 443-453 (1970)) using default parameters (i.e., match=+1, mismatch=−1, and gap=−1). Then, the number of matches is counted for the optimal alignment. Finally, the complementarity fraction is defined as the number of matches divided by the smaller of the lengths of either sequence, i.e., the fraction of the length that is complementary. The term “substantially complementary” includes completely complementary nucleic acid strands.

A “tile” refers to one or more contiguous loci within a region of interest. A region of interest can be divided into one or more tiles. The tiles can be, but need not be, contiguous. Therefore, the region of interest can optionally include non-contiguous sub-regions. The tiles can be of the same length or of different lengths. A “locus” refers to one or more contiguous bases, and is fully contained within the tile.

Where a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

### Balanced Capture Probe Libraries

Preparation of a balanced capture probe library includes sequencing one or more sequencing libraries comprising a plurality of nucleic acid molecules enriched using a first (e.g., unbalanced) capture probe library comprising a plurality of capture probes, each capture probe substantially complementary to a portion of or adjacent to a region of interest or a portion adjacent to the region of interest included in the sequencing library and an initial known amount used to form the first capture probe library. The aspects described in this section can be applied to any of the methods described herein, unless context clearly indicates otherwise.

The sequencing library comprises a plurality of nucleic acid molecules (which can also be referred to as “inserts” when attached to a sequencing adapter). In some embodiments the sequencing library comprises cell-free DNA (such as fetal cell-free DNA, tumor cell-free DNA, genomic cell-free DNA), fragmented DNA derived from cells in a sample (such as genomic DNA or mitochondrial DNA, which can be extracted from cells by lysing the cells and isolating the DNA contained therein). Fetal cell-free DNA circulates throughout the bloodstream of a pregnant woman, and is believed to originate primarily from the placental cells undergoing apoptosis during the pregnancy. Tumor cell-free DNA originates from tumor cells undergoing apoptosis in a subject with a tumor, and similarly circulates throughout the bloodstream of the subject. Tumor cell-free DNA and fetal cell-free DNA are generally a minor portion of the cell-free DNA circulating in the bloodstream, within the remaining cell-free DNA originating from non-tumor genomic sources of the subject (in the case of tumor cell-free DNA) or pregnant mother (in the case of fetal cell-free DNA). Cell-free DNA generally about 100 to about 500 bases in length. DNA can also be extracted and isolated from cells within patient samples (such as blood, saliva, tissue samples, etc.) and fragmented, for example by sonicating the genomic DNA. The size of the fragmented genomic DNA can vary depending on the method used for fragmentation, but is generally also about 100 bases to about 500 bases in length. In some embodiments, the sequencing library is an RNA sequencing library, which can be reverse transcribed either before or after enrichment. In some embodiments, the nucleic acid molecules in the sequencing library are ligated to sequencing adapters (at one or both ends), which optionally include molecular barcodes or sample index barcodes.

The nucleic acid molecules in an unenriched sequencing library can include fragments from throughout the genome, which is represented approximately uniformly by the nucleic acid molecules. Some variation in the represented genome can arise, for example, due to copy number variants or aneuploidies, or regions of genomic instability. The nucleic acid molecules in an unenriched sequencing library therefore represent the region of interest and regions of the genome other than the region of interest. In some embodiments, the region of interest comprises one or more chromosomes. In some embodiments, the region of interest comprises one or more genes (such as 2 or more, 3 or more, 4 or more, 5 or more, 10 or more, 15 or more, 20 or more, 30 or more, 40 or more, 50 or more, 75 or more, 100 or more, 150 or more, 200 or more, or 250 or more genes). In some embodiments, the region of interest comprises the exons of one or more genes (such as the exons from 2 or more, 3 or more, 4 or more, 5 or more, 10 or more, 15 or more, 20 or more, 30 or more, 40 or more, 50 or more, 75 or more, 100 or more, 150 or more, 200 or more, or 250 or more genes). In some embodiments, the region of interest comprises one or more exons (such as 2 or more, 3 or more, 4 or more, 5 or more, 10 or more, 15 or more, 20 or more, 30 or more, 40 or more, 50 or more, 75 or more, 100 or more, 150 or more, 200 or more, or 250 or more, 500 or more, 1000 or more, or 2000 or more exons). In some embodiments, the region of interest is contiguous. In some embodiments, the region of interest is region of interest is divided into one or more non-contiguous sub-regions or tiles.

Capture probe libraries can also be used for targeted genotyping, including targeting SNPs and indel variants. The region of interest can therefore be one or more bases, which need not be contiguous, within the genome. For example, in some embodiments, the region of interest comprises 1 or more non-contiguous sub-regions, 2 or more non-contiguous sub-regions, 3 or more non-contiguous sub-regions, 4 or more non-contiguous sub-regions, 5 or more non-contiguous sub-regions, 10 or more non-contiguous sub-regions, 25 or more non-contiguous sub-regions, 50 or more non-contiguous sub-regions, 100 or more non-contiguous sub-regions, 150 or more non-contiguous sub-regions, 200 or more non-contiguous sub-regions, or 250 more non-contiguous sub-regions. In some embodiments, each of the non-contiguous sub-regions comprises 1 or more contiguous bases, 2 or more contiguous bases, 3 or more contiguous bases, 4 or more contiguous bases, or 5 or more contiguous bases. For example, in some embodiments each of the non-contiguous sub-regions comprises 1 to about 20 contiguous bases (such as 1 to about 10 contiguous bases, or about 1 to about 5 contiguous bases).

In some embodiments, the initial known amount of each capture probe is an initial known volume of the capture probe, an initial known mass of the capture probe, or an initial known number of moles of the capture probe. The initial known amount can also be an initial known relative amount (i.e., fraction or percentage) of each capture probe in the capture probe library. The relative amount can be a relative known volume, mass, or number of moles. The capture probe library is generally formed by combining the capture probes in a solution. In some embodiments, each capture probe is provided in a stock solution, and the stock solution is combined to form the capture probe library. The exact concentration of the stock solution is often not known, or the concentration is known within a significant error. However, the concentration of the stock solution need not be known to form the balanced capture probe library, for example when the same stock solution is used to form the first capture probe library and any subsequent, balanced capture probe library. Instead, the relative amount (e.g., volume) of each capture probe can be adjusted to construct the balanced capture probe library. The subsequent known amount of each capture probe in the balanced capture probe depends, in part, on the initial known amount of each capture probe, and can be determined in the same amount type (i.e., volume, number of moles, grams, concentration, etc.).

The sequencing library can be enriched for the region of interest by isolating (and optionally amplifying) nucleic acid molecules that include portions of the region of interest using a capture probe library. That is, the capture probe library can be combined with the sequencing library (which includes the region of interest) to enrich the sequencing library for nucleic acid molecules comprising portions of the region of interest. Methods for enriching sequencing libraries using capture probes are generally known in the art, and can include hybrid capture methods (e.g., using biotinylated capture probes), PCR amplification using capture probes as PCR primers, and direct targeted sequencing (also referred to as “direct capture sequencing”) see, for example, U.S. Patent Publication No. 2014/0162278; U.S. Pat. No. 9,092,401; and U.S. Pat. No. 9,309,556), as described in further detail below. See also Ng et al., Targeted Capture and Massively Parallel Sequencing of Twelve Human Exomes, Nature vol. 461 (7261), pp. 272-276 (2009).

The capture probes in the capture probe library are nucleic acid oligonucleotides that can be used to enrich the region of interest. The capture probes are designed to hybridize to nucleic acid molecules containing a portion of the region of interest. The capture probes are therefore substantially complementary to a portion of the region of interest or substantially complementary to a portion adjacent to the region of interest. Some of the nucleic acid molecules in the sequencing library include a portion of the region of interest, and can hybridize to the capture probes. Some of the nucleic acid molecules may also include portions of the genome that are adjacent to the region of interest, which may hybridize to capture probes that are substantially complementary to those portions.

In some embodiments, hybrid capture methods are used to enrich the region of interest by combining capture probes that are substantially complementary to a portion of the region of interest with the sequencing library, thereby hybridizing the capture probes to nucleic acid molecules comprising the portion of the region of interest. The nucleic acid molecules that hybridize to the capture probes can be isolated from non-hybridized nucleic acid molecules (for example, by pull-down methods). The hybridized complex can be denatured and the enriched nucleic acid molecules from the sequencing library can be sequenced. In some embodiments, the enriched nucleic acid molecules are re-enriched in a second (or more) round of hybridization to the capture probes, isolation and denaturation before being sequenced. Optionally, the nucleic acid molecules in the sequencing library can be amplified (for example, by PCR) either before or after enrichment.

In some embodiments, such as when direct targeted sequencing methods are used, capture probes that are substantially complementary to a portion of the region of interest or substantially complementary to a portion adjacent to the region of interest are combined with the sequencing library, thereby hybridizing the capture probes to the nucleic acid molecules comprising to the portion of the region of interest or to the portion adjacent to the region of interest. In direct targeted sequencing methods, the capture probe is extended using the nucleic acid molecule as a template, and the extended capture probe is sequenced. Since the extended capture probe (or amplified copies of the extended capture probe) itself is sequenced, the sequence of the capture probe is not interpreted as the sequence arising from the sample, although it can be used to aid sequence alignment. Additionally, in some embodiments, one or more capture probes binds to a portion adjacent to the region of interest, which can assist in sequencing the 5′ end of a region of interest (which can also be sequenced by sequencing the 3′ end of the complementary strand).

In some embodiments, one or more of the capture probes are attached to an additional oligonucleotide (such as a primer binding site or other specialized nucleic acid segment). In some embodiments, the capture probes in the capture probe library are DNA oligonucleotides, RNA oligonucleotides, or a mixture of DNA oligonucleotides and RNA oligonucleotides. In some embodiments, the capture probes are about 12 to about 300 bases in length (such as about 12 bases to about 20 bases, 20 bases to about 60 bases in length, about 60 bases to about 100 bases in length, or about 100 bases to about 160 bases, about 160 bases to about 220 bases, or about 220 bases to about 300 bases in length).

In some embodiments two or more capture probes in the capture probe library are substantially complementary to an overlapping segment of the region of interest. That is, in some embodiments, a given segment of the region of interest can hybridize to two or more capture probes. In some embodiments, the two or more capture probes hybridize to the same strand of the nucleic acid molecule. In some embodiments, the two or more capture probes hybridize to different strands of the nucleic acid molecule. In some embodiments, none of the capture probes in the capture probe library are substantially complementary to an overlapping segment of the region of interest.

The number of capture probes in the capture probe library can depend on the size of the region of interest, as a larger region of interest generally requires a larger number of capture probes for adequate coverage. In some embodiments, the capture probe library comprises about 10 or more unique capture probes (such as about 50 or more, about 100 or more, about 250 or more, about 500 or more, about 1000 or more, about 2500 or more, about 5000 or more, about 10,000 or more, about 25,000 or more, about 50,000 or more, about 100,000 or more, or about 200,000 or more) unique capture probes.

The enriched sequencing library can be sequenced using a high-throughput sequencer, such as an Illumina HiSeq2500, Illumina HiSeq3000, Illumina HiSeq4000, Illumina HiSeqX, Roche 454, or Life Technologies Ion Proton sequencing systems can also be used. Other methods of sequencing are known in the art.

Sequencing the enriched sequencing library generates a plurality of sequencing reads. In some embodiments, the sequencing reads are aligned to a reference sequence (such as a reference genome or a reference region of interest). In some embodiments, the sequencing depth of a particular locus is based on the number of sequencing reads that align at that locus. In some embodiments, the sequencing reads are aligned to the capture probes. In some embodiments, the sequencing depth is based on the number of sequencing reads that align to the capture probe. In some embodiments, the sequencing depth is adjusted to correct for GC bias. In some embodiments, the sequencing depth is adjusted to correct for mappability. In some embodiments, the sequencing reads are adjusted to account for PCR duplicates that arise from PCR amplification of a sequencing library prior to ligating the nucleic acid molecules to sequencing adapters by eliminating duplicate reads.

In some embodiments, the sequencing depth is a raw number of sequencing reads (which may be adjusted to correct for GC bias, mappability, PCR duplicates, or other sequencing artifacts). In some embodiments, the sequencing depth is based on a number of collapsed consensus sequences. Collapsed consensus sequences are built by identifying sequencing reads arising from the same parent nucleic acid molecule in a sequencing library (for example, those sequencing reads that include a common molecular barcode and that align a common reference sequence). A molecular barcode can be included in a sequencing adapter ligated to an insert nucleic acid molecule in the sequencing library. Nucleic acid molecules that are amplified retain the same molecular barcode. After sequencing the amplified nucleic acid molecules, the generated sequencing reads can be traced to the parent (i.e., pre-amplified) nucleic acid molecule based on the common molecular barcode and alignment of the sequencing reads. A consensus sequence can be built based on the aligned sequencing reads with the common molecular barcode. The individual sequencing reads are said to be “collapsed” into the consensus sequence because the consensus sequence represents each of the sequencing reads that arise from the same parent nucleic acid molecule. A sequencing depth calculated using a collapsed sequencing depth is the number of collapsed consensus sequences (and thus, the number of represented parent nucleic acid molecules) at any locus. In some embodiments, the sequencing depth is based on a number of duplexed collapsed consensus sequences. Duplexed collapsed consensus sequences are pairs of collapsed consensuses sequences which include both strands of the parent nucleic acid molecule.

The capture probe library is constructed by combining the capture probes. The balanced capture probe library is constructed by combining the capture probes (or at least a fraction of the capture probes) at the subsequent amounts as determined by the methods described herein. In some embodiments, the capture probes are mixed in solution (such as an aqueous buffer). The capture probes can be combined, for example, by a liquid handler or a pipetting system (which may be manual or automatic). In some embodiments, the liquid handler or the pipetting system can handle volumes in a wide range, for example about 50 nL to about 1 mL (or about 100 nL to about 500 μL).

### Balanced Capture Probe Libraries for Hybridized Capture Methods

A balanced capture probe library can be constructed to enrich a sequencing library to minimize the difference between a predicted sequencing depth profile and a desired sequencing depth profile. The desired sequencing depth profile can be uniform or non-uniform, and the desired sequencing depth profile can be implied by an objective function used to minimize the difference between the predicted and desired sequencing depth profiles. In some embodiments, construction of the balanced capture probe library is constrained by a minimum amount or a maximum amount of the capture probes in the balanced capture probe library.

In some embodiments, the method preparing a balanced capture probe library comprises sequencing a sequencing library (or a plurality of sequencing libraries) comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; selecting a subsequent known amount of each capture probe based on the initial known amount and the sequencing depth attributable to said capture probe, wherein the subsequent known amount of each capture probe is selected to minimize a difference between a predicted sequencing depth profile and a desired sequencing depth profile; and constructing the balanced capture probe library by combining at least a fraction of the capture probes at the subsequent known amount for each capture probe in the balanced capture probe library. In some embodiments, the desired sequencing depth profile is non-uniform. In some embodiments, minimization of the difference is constrained by a minimum subsequent amount or a maximum subsequent amount of each capture probe.

The sequencing library can be enriched by combining the capture probe library with the sequencing library and isolating the nucleic acid molecules in the sequencing library that hybridize to any one or more of the capture probes in the capture probe library. For example, in some embodiments, capture probes are bound by a purification moiety (such as biotin), and the nucleic acid molecules from the sequencing library that include a segment of the region of interest can be isolated by hybrid capture methods. Nucleic acid molecules form the sequencing library that hybridize to the capture probe can be isolated by pulling down the capture probe bound to the purification moiety, thereby simultaneously pulling down nucleic acid molecules from the sequencing library that comprise a segment that is substantially complementary to the capture probe. In some embodiments, the capture probes are biotinylated. Nucleic acid molecules that hybridize to the biotinylated capture probes can be isolated by pulling down the biotinylated capture probes, for example by using a streptavidin conjugated surface (such as streptavidin beads, which can be magnetic).

The sequencing depth attributable to each capture probe is based on the number of sequencing reads that align to at least a portion of the capture probe. In some embodiments, the sequencing reads are consensus sequences formed by collapsing all sequencing reads originating from a parent nucleic acid molecule, which can be identified through a molecular barcode (i.e., collapsed consensus sequences). In some embodiments, the sequencing reads are duplex collapsed consensus sequences. In some embodiments, the sequencing reads are aligned using a reference sequence, and sequencing reads are attributed to a capture probe that uniquely aligns with at least a portion of the sequencing read. In some embodiments, the sequencing reads are directly aligned with the capture probes, and a sequencing read that uniquely aligns with the capture probe is attributed to that capture probe. The number of sequencing reads comprising a segment that is substantially complementary to at least a portion of the capture probe can then be designated as the sequencing depth attributable to that capture probe. In some embodiments, the sequencing depth is based on a number of sequencing reads that comprises a segment that is substantially complementary to at least 20%, at least 30%, at least 40%, at least 50%, at least 60%, at least 70%, at least 80%, or at least 90% of the capture probe. That is, in some embodiments, sequencing reads that are not substantially complementary for at least 20%, at least 30%, at least 40%, at least 50%, at least 60%, at least 70%, at least 80%, or at least 90% of the capture probe are excluded from the sequencing depth.

Once the sequencing depth attributable to a given capture probe is determined, it is possible to use the sequencing depth to obtain a binding fraction (that is, a determined fraction of the nucleic acid molecules in the sequencing library that comprise a segment substantially complementary to a sequence within the capture probe that bound to (i.e., hybridized to) the capture probe during the enrichment of the sequencing library) for the capture probe. In some embodiments, the segment substantially complementary to the sequence within the capture probe comprises at least 20%, at least 30%, at least 40%, at least 50%, at least 60%, at least 70%, at least 80%, or at least 90% of the capture probe. Preferably, the number of capture probe molecules is in excess relative to the number of nucleic acid molecules comprising a segment corresponding to at least a portion of the capture probe.

It is assumed that the nucleic acid molecules in the sequencing library are in binding equilibrium with the capture probes during the enrichment step. Therefore, a given capture probe (Pᵢ) and the set of nucleic acid molecules (Fᵢ) comprising a segment substantially complementary to a sequence within the capture probe are in a binding equilibrium with a complex (PᵢFᵢ) of both the binding probe and the capture probe as follows:

Pᵢ+Fᵢ [symbol] PᵢFᵢ

The binding constant (Kᵢ) for each capture probe is defined by:

$${K}_{i} = \frac{[ {P}_{i} {F}_{i} ]}{[ {P}_{i} ] [ {F}_{i} ]}$$

The total concentration of nucleic acid molecules (Fᵢ^{total}) in the sequencing library comprising a segment substantially complementary to the sequence within the capture probe (regardless of whether it is bound to the capture probe or not) is determined by.

[Fᵢ^{total}]=[Fᵢ]+[PᵢFᵢ]

Therefore, the free unbound nucleic acid molecule is:

[Fᵢ]=[Fᵢ^{total}]−[PᵢFᵢ]

The equation used to determine the total concentration of the nucleic acid molecule (Fᵢ^{total}) can be combined with the equation used to determine the binding constant (Kᵢ) as follows:

[PᵢFᵢ]=Kᵢ[Pᵢ]([Fᵢ^{total}]−[PᵢFᵢ])

This equation can be arranged as:

$$[ {P}_{i} {F}_{i} ] = [ {F}_{i}^{total} ] {( 1 + \frac{1}{{K}_{i} [ {P}_{i} ]} )}^{- 1}$$

Although the product of Kᵢ [Pᵢ] cannot be directly observed, the product can be defined as the effective concentration (cᵢ) of the capture probe (Pᵢ):

cᵢ=Kᵢ[Pᵢ]

The binding fraction for capture probe i can be defined as πᵢ according to:

$${π}_{i} = \frac{[ {P}_{i} {F}_{i} ]}{[ {P}_{i} {F}_{i} ] + [ {F}_{i} ]} = {( 1 + \frac{1}{{K}_{i} [ {P}_{i} ]} )}^{- 1}$$

The sequencing depth attributable to a given capture probe (i) in a given sequencing library (indexed by s) can be assumed proportional to the concentration of nucleic acid molecules from the sample comprising a segment substantially complementary to a sequence within the capture probe that are bound (i.e., hybridize) to the capture probe during the enrichment step. This can be demonstrated by the following equation:

dᵢₛ∂[PᵢFᵢ]ₛ

The assumption that the sequencing depth is proportional to the concentration of nucleic acid molecules that bind to the capture probe is justified because non-bound nucleic acid molecules are not retained during the enrichment step (i.e., they are washed away, not amplified, or otherwise not enriched). Thus, only those nucleic acid molecules that bind to the capture probe during the enrichment step are sequenced during downstream steps of the method.

It is also assumed that the total concentration of nucleic acid molecules in the sample [Fᵢₛ^{total}] that are substantially complementary to a sequence within each capture probe is constant and does not depend on the particular probe location within the genome. That is, all portions of the region of interest are assumed to be evenly represented by the nucleic acid molecules in the sequencing library. Therefore, the total amount of nucleic acid molecules (Nₛ) from the sequencing library is proportional to the total concentration of nucleic acid molecules [Fᵢₛ^{total}] that comprises a segment substantially complementary to a sequence within each capture probe:

Nₛ∂[Fᵢₛ^{total}]

Using the equations above, the sequencing depth attributed to each capture probe is related to the binding fraction according to the following equation:

dᵢₛ∂[PᵢFᵢ]ₛ∂Nₛπᵢ

The proportionality constant in this relationship can be absorbed by the amount of nucleic acid molecules in the sequencing library (Nₛ). The binding fraction πᵢ is probe dependent, but independent of the number of nucleic acid molecules in the sequencing library when the capture probe is in excess. Additionally, the determined sequencing depth will include some amount of noise, and the proportionality equation of the sequencing depth will be satisfied on average, given a large number of samples. That is:

[symbol]

dᵢ [symbol] =Nₛπᵢ

It is therefore preferable that a plurality of sequencing libraries be used to prepare the balanced capture probe library. Further, the sequencing depth for a given capture probe can be approximated as a Poisson distribution:

dᵢₛ=Poisson(Nₛπᵢ)

Once the sequencing depth attributable to each capture probe is determined (the sequencing depth can be based on the number of sequencing reads that comprise a segment substantially complementary to a sequence within the capture probe), a balanced capture probe library can be constructed by combining the at least a portion of capture probes at a subsequent known amount (that is, subsequent to the amount used in the first capture probe library). Not all capture probes in the first capture probe library need to be used in the balanced capture probe library as long as, if present, each capture probe is present in the subsequent amount (or relative amount). The subsequent known amount is based on the initial known amount and the sequencing depth attributable to each capture probe at that initial known amount. In some embodiments, the method includes obtaining for each capture probe a binding fraction of the nucleic acid molecules comprising a segment that is substantially complementary to a sequence within the capture probe that bound to the capture probe during enrichment of the sequencing library based on the sequencing depth attributable to the capture probe. For example, in some embodiments, the binding fraction πᵢ is obtained as described above. In some embodiments, the subsequent known amount of the capture probe in the balanced capture probe library is based on the initial known amount and the binding fraction.

The sequencing depth attributable to each capture probe can vary as a function of the effective concentration of the capture probe, which depends on the amount of the capture probe included in the capture probe library. The effective concentration of a given capture probe in the first capture probe library (which is based on the initial amount of the capture probe in the capture probe library) can be multiplied by a coefficient μᵢ to obtain a sequencing depth as a function of the coefficient:

$${d}_{is} ( {μ}_{i} ) = {{N}_{s} ( 1 + \frac{1}{{μ}_{i} {c}_{i}} )}^{- 1}$$

The determined sequencing depth attributable to a given capture probe i using the first capture probe library can be used to determine the initial effective concentration cᵢ, which can be adjusted according to the coefficient μᵢ to achieve the desired level of balance between the plurality of capture probes in the capture probe library.

The concentration of the capture probe in the balanced capture probe library [Pᵢ′] can be related to the concentration of the capture probe in the first capture probe library [Pᵢ] through the initial known amount of the capture probe yi and the subsequent known amount of the capture probe vᵢ. The initial and subsequent known amounts of the capture probe can be a volume, a mass, or a number of moles, or an initial and subsequent known relative amount. The relationship between the concentration of the capture probe in the balanced capture probe library and the concentration of the capture probe in the first capture probe library is related according to:

$$[ {P}_{i}^{′} ] = \frac{{v}_{i}}{{γ}_{i}} [ {P}_{i} ]$$

The relationship between the capture probe concentration and the initial and subsequent amounts of the capture probe can be used to relate the binding fraction πᵢ of the nucleic acid molecules in the sequencing library comprising a segment substantially complementary to at least a portion of the capture probe that bound to the capture probe according to:

$${π}_{i} ( {v}_{i} ) = {( 1 + \frac{{γ}_{i}}{{v}_{i} {c}_{i}} )}^{- 1}$$

Sequencing depth attributable to the capture probe in a balanced capture probe library can then be predicted as a function of the subsequent known amount of the capture probe in the balanced capture probe library based on the initial known amount of the capture probe in the first capture probe library and the binding fraction of the nucleic acid molecules comprising a segment substantially complementary to at least a portion of the capture probe that bound to the capture probe during the enrichment of the sequencing library (which can be obtained from the sequencing depth attributable to the capture probe in the first capture probe library) according to:

$${d}_{is} ( {v}_{i} ) = {N}_{s} {π}_{i} ( {v}_{i} ) = {{N}_{s} ( 1 + \frac{{γ}_{i}}{{v}_{i} {c}_{i}} )}^{- 1}$$

The subsequent amount vᵢ for each capture probe in the balanced capture probe library can be selected to minimize the difference between a predicted sequencing depth profile and a desired sequencing depth profile, which is optionally non-uniform. A uniform desired sequencing depth profile would require the sequencing depth attributable to each capture probe in the balanced capture probe library to be the same. Thus, a non-uniform desired sequencing depth profile requires the sequencing depth attributable to at least one capture probe in the balanced capture probe library to be different from the sequencing depth attributable to at least one other capture probe in the capture probe library. For example, the difference may be a two-fold or more, three-fold or more, four-fold or more, or five-fold or more increase or decrease in sequencing depth. In some embodiments, the desired sequencing depth profile is uniform.

The difference between the predicted sequencing depth profile and the desired sequencing depth profile can be minimized based on any objective function, such as a coefficient of variation (i.e., the ratio of a measure of variation (such as a standard deviation) to an average (such as a mean)), using the predicted sequencing depth dᵢₛ(vᵢ) or binding fraction πᵢ. The difference between the predicted sequencing depth profile and the desired sequencing depth profile can also be minimized based on an objective function using the effective concentration (cᵢ), for example by defining the objective function as an implicit function of the effective concentration.

Since the number of nucleic acid molecules in the sequencing library is independent of the capture probe, minimizing the difference between the predicted sequencing depth profile and the desired depth profile can be accomplished by minimizing the variability of the binding fraction πᵢ between the capture probes in the capture probe library. Therefore, Nₛ can be ignored when minimizing the variability. The difference can be defined by an objective function of the subsequent amounts of the capture probes in the balanced capture probe library, according to:

$$f ( \vec{v} ) = V ( \vec{π} ( \vec{v} ) )$$

$$wherein : {( \vec{π} ( \vec{v} ) )}_{i} = {( 1 + \frac{{γ}_{i}}{{v}_{i} {c}_{i}} )}^{- 1}$$

and wherein V( ) is a user-defined objective function (such as the coefficient of variation) for the capture probe library, (\vec{π}(\vec{v}))ᵢ is the binding fraction of the nucleic acid molecules comprising a segment substantially complementary to at least a portion of the capture probe i that bound to the capture probe i during enrichment of the sequencing library for the vector \vec{π}(\vec{v}), cᵢ is an effective concentration for capture probe i for the vector \vec{π}(\vec{v}), vᵢ is the subsequent amount of capture probe i for the vector \vec{π}(\vec{v}); and γᵢ is the initial amount of capture probe i for the vector \vec{π}(\vec{v}).

Alternatively, minimizing the difference between the predicted sequencing depth profile and the desired sequencing depth profile can comprise minimizing an objective function for the effective concentration cᵢ instead of πᵢ.

The binding fraction πᵢ can be obtained based on the determined sequencing depth attributable to the capture probe by fitting a model, such as a maximum likelihood model or a Markov chain Monte Carlo model. For example, a maximum log-likelihood (LL) model can be fit for a capture probe i in the sequencing library s according to:

$$LL = {\sum }_{is} {d}_{is} log ( {N}_{S} {π}_{i} ) - {N}_{s} {π}_{i}$$

The desired sequencing depth can be implied by the objective function. For example, the objective function can assume the desired sequencing depth to be uniform by applying uniform objective function terms to all capture probes. For example, by minimizing the coefficient of variation of sequencing depth across all capture probes without further adjustment, the desired sequencing profile is implied to be uniform. The desired sequencing depth profile therefore need not be explicitly defined. In some embodiments, the non-uniform sequencing depth profile comprises a first sequencing depth (or relative sequencing depth) attributable to a first capture probe in the capture probe library and a second sequencing depth (or relative sequencing depth) attributable to a second capture probe in the capture probe library. The objective function can be a summed statistical difference (such as a sum of squares of the difference) between the predicted sequencing depth and the desired sequencing depth attributable to the capture probes, wherein the desired sequencing depth is defined to be non-uniform. Minimization of the objective function can then provide the subsequent amounts of the capture probes in the capture probe library.

In some embodiments, minimization of the difference between the predicted sequencing depth profile and the desired sequencing depth profile is constrained by a minimum subsequent amount or a maximum subsequent amount (or both a minimum and a maximum subsequent amount) of each capture probe. This may be done to avoid making very large changes in the amount of each probe in the capture probe library between the first capture probe library and the balanced capture probe library. Further, dispensing very small amounts of the capture probe are subject to precision limits. For example, pipetting nanoliter volumes is impractical without some amount of error. Additionally, there may be finite amounts of the capture probe available, thereby limiting the amount of capture probe that can be used to form the capture probe library. A very large amount of a single probe could also result in undesirable dilution of the other capture probes in the capture probe library. By constraining the minimum and/or maximum amounts of each capture probe used to form the library during the minimization determination, dramatic changes can be minimized (which can minimize unaccounted for interactions) and practical amounts of each capture probe can be combined to form the capture probe library. This optimization can be written as:

$${minimize}_{v} f ( \vec{v} )$$

$$subject to m \leq {v}_{i} \leq M$$

wherein m is the minimum subsequent amount of the capture probe and M is the maximum subsequent amount of the capture probe. In some embodiments, the minimum subsequent amount is about 10 nanoliters (nL) or more (such as about 20 nL or more, about 30 nL or more, about 40 nL or more, about 50 nL or more, about 75 nL or more, or about 100 nL or more). In some embodiments, the maximum subsequent amount is about 1 mL or less (such as about 750 μL or less, about 500 μL or less, about 400 μL or less, about 300 μL or less, about 200 μL or less, about 100 μL or less, about 75 μL or less, about 50 μL or less, about 25 μL or less, or about 10 μL or less).

Once the subsequent amounts of each capture probe are determined, the second (i.e., “balanced”) capture probe library can be constructed by combining the capture probes at the subsequent known amount (such as volume, mass, or number of moles, as for the initial known amount).

The method of capture probe balancing can optionally be performed iteratively. That is, the second (balanced) capture probe library can be re-balanced one or more times, two or more times, three or more times, four or more times, or five or more times. Preferably, the same sequencing library used to initially balance the capture probe library is used to rebalance the capture probe library. In some embodiments, the method further comprises enriching the sequencing library using the balanced capture probe library; sequencing the sequencing library enriched using the balanced capture probe library; determining a sequencing depth attributable to each capture probe in the balanced capture probe library; selecting a second subsequent known amount of each capture probe based on the subsequent known amount of said capture probe in the balanced capture probe library and the sequencing depth attributable to said capture probe, wherein the second subsequent known amount of each capture probe is selected to minimize a difference between a second predicted sequencing depth profile and the desired sequencing depth profile; and constructing a re-balanced capture probe library by combining at least a fraction of the capture probes at the second subsequent known amount of each capture probe in the re-balanced capture probe library. In some embodiments, minimization of the difference is constrained by a minimum second subsequent amount or a maximum second subsequent amount of each capture probe. The minimum subsequent amount or the maximum subsequent amount may be the same as or different from the minimum subsequent amount or the maximum subsequent amount used in the first or any late re-balancing iteration.

The balanced capture probe library can be used to enrich a test sequencing library. For example, in some embodiments, a method of enriching a test sequencing library comprises (1) combining the test sequencing library comprising nucleic acid molecules with the capture probe library prepared by sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; selecting a subsequent known amount of each capture probe based on the initial known amount and the sequencing depth attributable to said capture probe, wherein the subsequent known amount of each capture probe is selected to minimize the difference between a predicted sequencing depth profile and a desired sequencing depth profile; and constructing the balanced capture probe library by combining at least a fraction of the capture probes at the subsequent known amount for each capture probe in the balanced capture probe library; and (2) selecting nucleic acid molecules from the test sequencing library that hybridize with the capture probes in the capture probe library. In some embodiments, the desired sequencing depth profile is non-uniform. In some embodiments, minimization of the difference is constrained by a minimum subsequent amount or a maximum subsequent amount of each capture probe.

In some embodiments the enriched test sequencing library is sequenced. In some embodiments the enriched test sequencing library is amplified prior to sequencing the enriched test sequencing library, for example by polymerase chain reaction (PCR) or bridge amplification on a sequencing flow cell (such as an Illumina flow cell). In some embodiments, the capture probes are removed from the test sequencing library after enrichment, for example by fixing the nucleic acid molecules in the enriched sequencing library to a surface (such as a flow cell) and washing away the capture probes.

Two or more balanced (including rebalanced) capture probe libraries can be combined to form a pooled capture probe library. In some embodiments, the region of interest for each balanced capture probe library is distinct.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; selecting a subsequent known amount of each capture probe based on the initial known amount and the sequencing depth attributable to said capture probe, wherein the subsequent known amount of each capture probe is selected to minimize a difference between a predicted sequencing depth profile and a desired sequencing depth profile; and constructing the balanced capture probe library by combining at least a fraction of the capture probes at the subsequent known amount of each capture probe in the balanced capture probe library. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; selecting a subsequent known amount of each capture probe based on the initial known amount and the sequencing depth attributable to said capture probe, wherein the subsequent known amount of each capture probe is selected to minimize a difference between a predicted sequencing depth profile and a desired sequencing depth profile, and wherein minimization of the difference is constrained by a minimum subsequent amount or a maximum subsequent amount of each capture probe; and constructing the balanced capture probe library by combining at least a fraction of the capture probes at the subsequent known amount of each capture probe in the balanced capture probe library. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; selecting a subsequent known amount of each capture probe based on the initial known amount and the sequencing depth attributable to said capture probe, wherein the subsequent known amount of each capture probe is selected to minimize a difference between a predicted sequencing depth profile and a non-uniform desired sequencing depth profile, and wherein minimization of the difference is constrained by a minimum subsequent amount or a maximum subsequent amount of each capture probe; and constructing the balanced capture probe library by combining at least a fraction of the capture probes at the subsequent known amount of each capture probe in the balanced capture probe library. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; selecting a subsequent known amount of each capture probe based on the initial known amount and the sequencing depth attributable to said capture probe, wherein the subsequent known amount of each capture probe is selected to minimize a difference between a predicted sequencing depth profile and a non-uniform desired sequencing depth profile; and constructing the balanced capture probe library by combining at least a fraction of the capture probes at the subsequent known amount for each capture probe in the balanced capture probe library. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; obtaining for each capture probe a binding fraction based on the sequencing depth attributable to the capture probe; selecting a subsequent known amount of each capture probe based on the initial known amount and the binding fraction, wherein the subsequent known amount of each capture probe is selected to minimize a difference between a predicted sequencing depth profile and a desired sequencing depth profile; and constructing the balanced capture probe library by combining at least a fraction of the capture probes at the subsequent known amount of each capture probe in the balanced capture probe library. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; obtaining for each capture probe a binding fraction based on the sequencing depth attributable to the capture probe; selecting a subsequent known amount of each capture probe based on the initial known amount and the binding fraction, wherein the subsequent known amount of each capture probe is selected to minimize a difference between a predicted sequencing depth profile and a desired sequencing depth profile, and wherein minimization of the difference is constrained by a minimum subsequent amount or a maximum subsequent amount of each capture probe; and constructing the balanced capture probe library by combining at least a fraction of the capture probes at the subsequent known amount of each capture probe in the balanced capture probe library. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; obtaining for each capture probe a binding fraction based on the sequencing depth attributable to the capture probe; selecting a subsequent known amount of each capture probe based on the initial known amount and the binding fraction, wherein the subsequent known amount of each capture probe is selected to minimize a difference between a predicted sequencing depth profile and a non-uniform desired sequencing depth profile, and wherein minimization of the difference is constrained by a minimum subsequent amount or a maximum subsequent amount of each capture probe; and constructing the balanced capture probe library by combining at least a fraction of the capture probes at the subsequent known amount of each capture probe in the balanced capture probe library. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; obtaining for each capture probe a binding fraction based on the sequencing depth attributable to the capture probe; selecting a subsequent known amount of each capture probe based on the initial known amount and the binding fraction, wherein the subsequent known amount of each capture probe is selected to minimize a difference between a predicted sequencing depth profile and a non-uniform desired sequencing depth profile; and constructing the balanced capture probe library by combining at least a fraction of the capture probes at the subsequent known amount for each capture probe in the balanced capture probe library. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; obtaining for each capture probe a binding fraction based on the sequencing depth attributable to the capture probe; selecting a subsequent known amount of each capture probe based on the initial known amount and the binding fraction, wherein the subsequent known amount of each capture probe is selected to minimize an objective function defining a difference between a predicted sequencing depth for each capture probe at the subsequent known amount; and constructing the balanced capture probe library by combining at least a fraction of the capture probes at the subsequent known amount of each capture probe in the balanced capture probe library. In some embodiments, the objective function is a coefficient of variation. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; obtaining for each capture probe a binding fraction based on the sequencing depth attributable to the capture probe; selecting a subsequent known amount of each capture probe based on the initial known amount and the binding fraction, wherein the subsequent known amount of each capture probe is selected to minimize an objective function defining a difference between a predicted sequencing depth for each capture probe at the subsequent known amount, and wherein minimization of the difference is constrained by a minimum subsequent amount or a maximum subsequent amount of each capture probe; and constructing the balanced capture probe library by combining at least a portion of the capture probes at the subsequent known amount of each capture probe in the balanced capture probe library. In some embodiments, the objective function is a coefficient of variation. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; obtaining for each capture probe a binding fraction based on the sequencing depth attributable to the capture probe; selecting a subsequent known amount of each capture probe based on the initial known amount and the binding fraction, wherein the subsequent known amount of each capture probe is selected to minimize an objective function defining a difference between a predicted sequencing depth for each capture probe at the subsequent known amount, and wherein minimization of the difference is constrained by a minimum subsequent amount or a maximum subsequent amount of each capture probe; and constructing the balanced capture probe library by combining at least a fraction of the capture probes at the subsequent known amount of each capture probe in the balanced capture probe library. In some embodiments, the objective function is a coefficient of variation. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining a sequencing depth attributable to each capture probe; obtaining for each capture probe a binding fraction based on the sequencing depth attributable to the capture probe; selecting a subsequent known amount of each capture probe based on the initial known amount and the binding fraction, wherein the subsequent known amount of each capture probe is selected to minimize an objective function defining a difference between a predicted sequencing depth for each capture probe at the subsequent known amount; and constructing the balanced capture probe library by combining at least a fraction of the capture probes at the subsequent known amount for each capture probe in the balanced capture probe library. In some embodiments, the objective function is a coefficient of variation. In some embodiments, the difference between the predicted sequencing depth profile and the desired sequencing depth profile is defined by an objective function. In some embodiments, the desired sequencing depth profile is implied by the objective function. In some embodiments, the initial known amount is an initial known volume and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth is based on a number of sequencing reads that align to a portion of the capture probe. In some embodiments, the sequencing depth is based on a number of sequencing reads that comprise a segment aligned to at least half of the capture probe. In some embodiments, the number of sequencing reads is a number of consensus sequencing reads. In some embodiments, the number of sequencing reads is a number of duplex consensuses sequencing reads. In some embodiments, the capture probes are not substantially complementary to overlapping portions of the region of interest. In some embodiments, at least two capture probes in the plurality of capture probes are substantially complementary to overlapping portions of the region of interest. In some embodiments, constructing the balanced capture probe library comprises combining each capture probe in the first capture probe library at the subsequent known amount of each capture probe in the balanced capture probe library. In some embodiments, enriching the sequencing library comprises separating nucleic acid molecules in the sequencing library that are hybridized to the capture probes from nucleic acid molecules in the sequencing library that are not hybridized to the capture probes.

Balanced Capture Probe Libraries for Enrichment by Extension of the Capture Probe

In another aspect, a balanced capture probe library can be constructed to enrich a sequencing library to obtain a desired sequencing depth profile at substantially all of a plurality of loci within the region of interest attributable to the balanced capture probe library. In some embodiments, the desired sequencing depth profile is a predetermined minimum sequencing depth. In some embodiments, the desired sequencing depth profile is uniform among substantially all of the loci in the plurality of loci. In some embodiments, the desired sequencing depth profile is non-uniform among substantially all of the loci in the plurality of loci. In some embodiments, a method of preparing a balanced capture probe library comprises sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining at each locus within a tile of the region of interest comprising one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the one or more contiguous loci within the tile; and constructing the balanced capture probe library by combining the at least a fraction of the capture probes at the subsequent known amount of each capture probe in the balanced capture probe library. In some embodiments, the sequencing depth profile is uniform. In some embodiments, the sequencing depth profile is non-uniform (for example, certain loci, tiles, or genes can have a higher desired sequencing depth than other loci, tiles, or genes). In some embodiments, the subsequent amount is selected to obtain a minimum sequencing depth at substantially all of the contiguous loci. In some embodiments, the subsequent amount is further selected to obtain a sequencing depth below a maximum sequencing depth at substantially all of the contiguous loci.

In some embodiments, the sequencing library is enriched by combining the capture probe library with the sequencing library and extending the capture probes that hybridize to the nucleic acid molecules in the sequencing library using the nucleic acid molecule as a template for the extension. The extended capture probes (with a complimentary copy of the hybridized nucleic acid molecule) can be sequenced (and optionally amplified prior to being sequenced). In some embodiments, the capture probes are extended using direct targeted sequencing. Exemplary methods of direct targeted sequencing are described in U.S. Patent Publication No. 2014/0162278; U.S. Pat. No. 9,092,401; and U.S. Pat. No. 9,309,556.

In some methods of enriching a sequencing library, capture probes in a capture probe library are bound to a solid surface, such as a sequencing flow cell. A sequencing library comprising a plurality of nucleic acid molecules flows across the flow cell, and the nucleic acid molecules comprising a portion of a region of interest complementary to the capture probes hybridize to the capture probes bound on the solid surface. The capture probes can then be extended using the hybridized nucleic acid molecule as a template. The extended capture probes bound to the surface therefore comprise a complementary portion of the region of interest included in the sequencing library, including the portion in the initial capture probe and the extended portion. The extended sequencing probes can be amplified (i.e., by bridge amplification) to form clusters of paired ends, which are then sequenced. Further details of this method are described in U.S. Patent Publication No. 2014/0162278; U.S. Pat. No. 9,092,401; and U.S. Pat. No. 9,309,556.

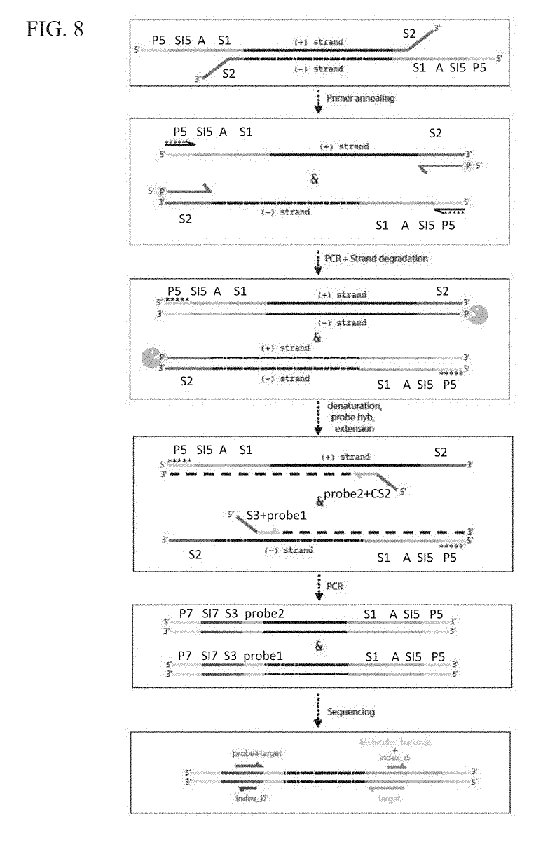

Although in some embodiments the capture probes are bound to a solid surface, in some embodiments the sequencing library is enriched in solution. For example, the sequencing library can be enriched using the method illustrated in FIG. 8. This method can be referred to as “solution direct targeted sequencing” or “solution-based direct targeted sequencing,” and is further detailed in U.S. Application No. 62/452,875 (filed Jan. 31, 2017), entitled “Methods and Compositions for Enrichment of Target Polynucleotides” and in U.S. Application No. 62/452,848 (filed Jan. 31, 2017), entitled “Methods and Compositions for Enrichment of Target Polynucleotides.” An adapter is ligated to each end of a nucleic acid duplex from the sequencing library. The nucleic acid duplex includes a first nucleic acid strand and a second nucleic acid strand, and the two strands are complementary to each other. The adapter includes: (i) a double stranded region that is ligated to one end of the target nucleic acid duplex; and (ii) first and second single stranded regions, wherein the first single stranded region includes an amplification primer binding sequence S2 and a 3′ end, and the second single stranded region comprises a 5′ end, a first grafting sequence (identified in FIG. 8 as “P5”) for attachment to a flow cell, an optional first sample index sequence (identified in FIG. 8 as “SI5”), an optional molecular barcode (identified in FIG. 8 as “A”), and a first sequencing primer binding site (identified in FIG. 8 as “S1”). The optional molecular barcode and the optional first sample index are preferably located between the first grafting sequence and the first sequencing primer binding site (in either order). The first grafting sequence is preferably located toward the 5′ end and the first sequencing primer binding site is preferably located toward the 3′ end of the adapter (that is, more proximal to the nucleic acid from the sample). The nucleic acid duplex ligated to the adapter can be amplified using a first amplification primer that includes a nucleic acid sequence that is complementary to the amplification primer binding sequence (identified in FIG. 8 as “S2”) and a second amplification primer that is complementary to at least a portion of the first grafting sequence (“P5”). The first amplification primer comprises a 5′ phosphate group, and the second amplification primer includes one or more nucleotide(s) at and/or near the 5′ end including a modification (such as a 2′-O-Me or a phosphorothioate group) to prevent nucleic acid strand degradation by an exonuclease enzyme (such as lambda exonuclease). The amplified nucleic acid can be digested using a 5′→3′ exonuclease (such as lambda exonuclease). Digestion of the amplified nucleic acids yields single stranded nucleic acid products that include the sequence of either the first or the second strand of the target nucleic acid duplex and the modified nucleotide(s) at and/or near the 5′ end. Capture probes are then hybridized to the nucleic acid molecules and extended. The capture probe includes a second sequencing primer binding sequence (identified in FIG. 8 as “S3”). The resulting primer extension products include the complement of the first grafting sequence (“P5”) at the 3′ end and the second sequencing primer binding sequence (“S3”) at the 5′ end. The amplified extension products are then amplified using (i) a third amplification primer that includes, from 5′ to 3″, a second grafting sequence (identified in FIG. 8 as “P7”), an optional second sample index sequence (identified in FIG. 8 as “SI7”), and a sequence that is complementary to the second sequencing primer binding sequence (“S3”); and (ii) a fourth amplification primer that includes the first grafting sequence (“P5”), thereby producing amplified primer extension products for sequencing.

Although sequencing is performed base-by-base, the bases can be grouped into loci (which include one or more contiguous bases, such as two, three, four, five or more contiguous bases) to lower the computational complexity. The sequencing depth at each locus can be the average or summed sequencing depth at the bases within the locus.

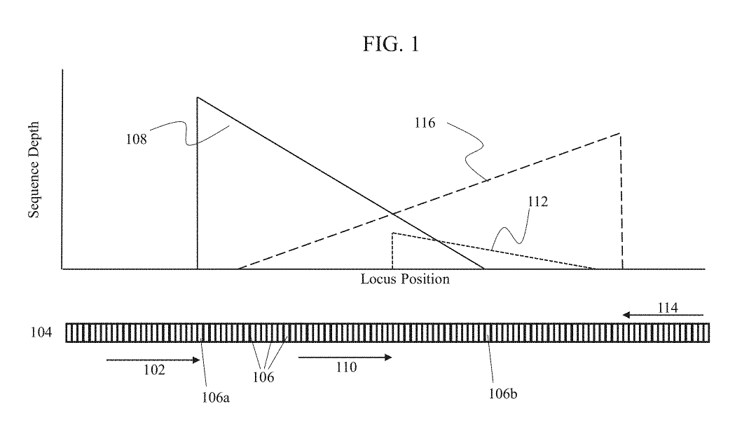

When the region of interest is enriched by extending the capture probes in the capture probe library, the sequencing depth (which may be a normalized sequencing read depth or a read depth corrected for GC bias or mappability) attributable to each capture probe at contiguous loci generally decreases (with some noise and variation) as a function of locus distance from the capture probe. Although the general trend is for decreased sequencing depth for loci more distant from the capture probe, for some sequencing probes the sequencing depth may increase before decreasing (or the sequencing depth may be relatively flat) as function of distance from the capture probe. A schematic of the sequencing depth attributable to several capture probes is shown in FIG. 1. The capture probe 102 was extended using a nucleic acid molecule from the sequencing library that hybridized to the capture probe. The region of interest 104 includes a plurality of contiguous loci 106 (small rectangles with only a portion of the individual loci being labeled). Capture probe 102 hybridizes to the region of interest 104 and is extended in the direction of the arrow. The sequencing depth in the example illustrated in FIG. 1A is high at the locus 106a most proximal to the capture probe 102, but decreases to a sequencing depth of about zero at locus 106b. The sequencing depth attributable to capture probe 102 is determined for each locus, and the sequencing depth profile 108 is plotted above. The determined sequencing depth for loci very distant or upstream of the capture probe 102 can be literally determined or assumed to be zero. A second capture probe 110 can be included in the capture probe library, which binds to a region of interest 104 at a location different from capture probe 102. The second capture probe 110 can be extended in the direction of the arrow and sequence, resulting in a second sequencing depth profile 112 attributable to capture probe 110. The second sequencing depth profile 112 can be different from the first sequencing depth profile 108. In some embodiments, the capture probe library can include a third capture probe 114, which can be a probe that binds the opposite strand of the region of interest as capture probe 102 and capture probe 110 and therefore extends in the opposite direction. A third sequencing depth profile 116 can be attributed to the third capture probe 114. A sequencing depth for one or more of the contiguous loci 106 can be determined that is attributable to each capture probe (i.e., capture probe 102, capture probe 110, and capture probe 114) as well as the capture probe library (i.e., the sum of the sequencing depth attributable to each of capture probe 102, capture probe 110, and capture probe 114).

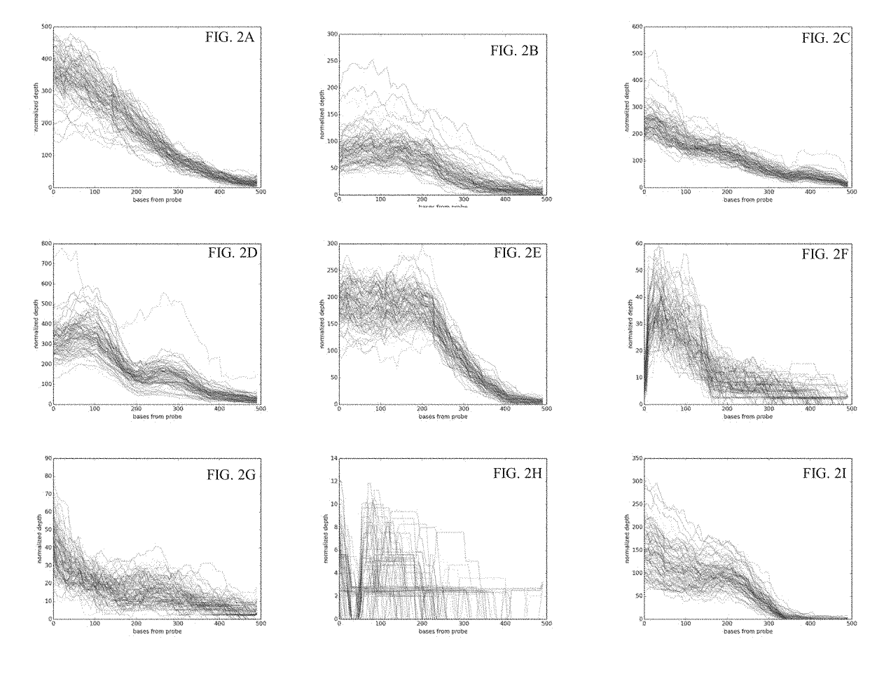

Exemplary types of sequencing depth profiles attributable to capture probes are shown in FIGS. 2A-I. Each of FIGS. 2A-I represents the sequencing depth profile of a single probe for 96 different samples. These profiles include FIG. 2A, which include sequencing depth profiles with exponential decay; FIG. 2B, which shows sequencing depth profiles that are highly variable from sample to sample, and have many increases and decreases in the sequencing depth as a function of distance from the capture probe, although with a general trend toward decreased sequencing depth; FIG. 2C, which include flat sequencing depth profiles and linear decay of sequencing depth; FIG. 2D, which include sequencing depth profiles with a humped pattern; FIG. 2E, which include plateaued sequencing depth profiles with an even sequencing depth for a first distance from the capture probe (to about 200 bases in the illustrated example) before a beginning a decay in sequencing depth; FIG. 2F, which include sequencing depth profiles that exhibit rapid decay of the sequencing depth in a short distance; FIG. 2G, which include sequencing depth profiles that have low sequencing depth close to the capture probe and decay with distance from the capture probe; FIG. 2H which include sequencing depth profiles with no or essentially no sequencing depth regardless of distance from the capture probe; and FIG. 2I, which include noise sequencing depth profiles that exhibit rapid decay of the sequencing depth in a short distance.

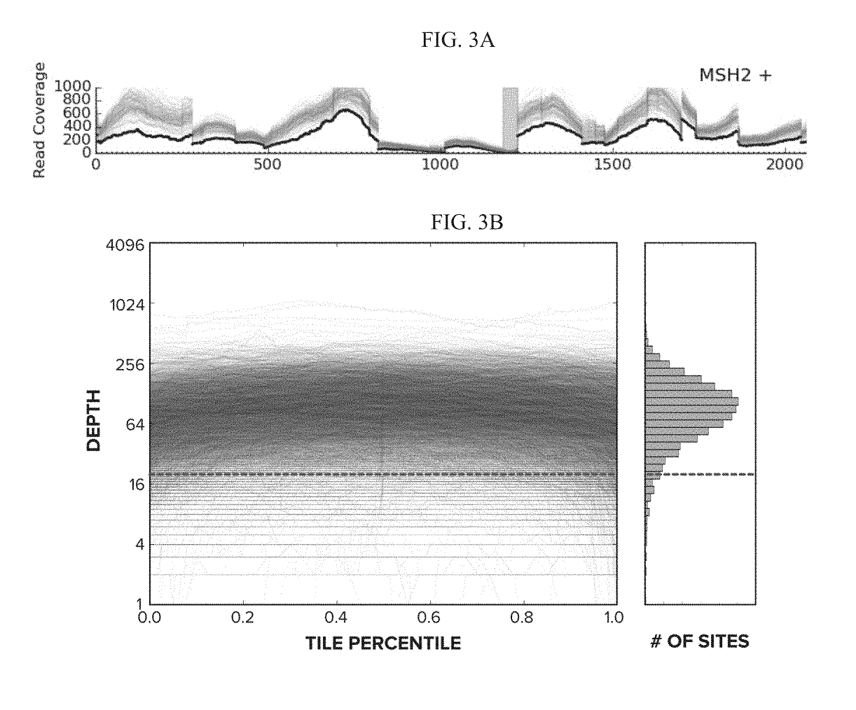

Because the various capture probes in the capture probe library perform differently and may be of different concentrations, the sequencing depth resulting from an unbalanced capture probe library is generally not uniform. For some probes, the sequencing depth may be insufficient or non-existent, yielding unreliable results. For some probes, the sequencing depth may be much higher than necessary, which gives diminishing returns. FIG. 3A illustrates sequencing depth of the MSH2 gene for 96 samples using more than 100 different capture probes in the capture probe library. The bold line represents the minimum number of sequencing reads obtained across any of the 96 samples. The shaded box indicates a portion of the gene with a sequencing depth lower than the predetermined minimum sequencing depth (20-fold coverage). Sequencing depth was capped at 1000-fold, resulting in some clipping at the top of the figure. As can be seen in FIG. 3A, the sequencing depth (read coverage) of the MSH2 gene is non-uniform, with certain portions of the gene have substantially greater sequencing depth than other portions of the gene. FIG. 3B illustrates sequencing depth across the >3500 tiles covering portions of >200 different genes using >12000 different capture probes. Though tiles can differ in length, for the figure, the lengths of all tiles are normalized to be equal, and the sequencing depth is plotted against the percentile of position within the tile. The dashed line indicates a predetermined minimum sequencing depth that is desired for a predetermined resolution in the sequencing data (20 sequencing reads at each position within a ROI tile in the illustrated case, although this number can be increased or decreased as desired). The right panel in FIG. 3B provides a histogram to better view the distribution of the sequencing depths shown in the left panel. While most loci shown in FIG. 3B have a sequencing depth above the minimum sequencing depth, some of the loci are have a sequencing depth threshold below the minimum.

When the amount of nucleic acid molecules in the sequencing library significantly exceeds the number of corresponding capture probes, the sequencing depth attributable to the capture probe is approximately proportional to amount of capture probe. Accordingly, sequencing depth for the corresponding loci can be increased by increasing the amount of the capture probe. However, simply increasing the amount of the entire capture probe library used to enrich the sequencing library would result in a substantial increase in the number of sequencing reads at loci for which additional sequencing reads is unnecessary. A better approach is to prepare a balanced capture probe library according to the methods described herein.

In some embodiments of preparing a balanced capture probe library, the method comprises sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining at each locus within a tile of the region of interest comprising one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the one or more contiguous loci; and constructing the balanced capture probe library by combining at least a fraction of the capture probes at the subsequent known amount of each capture probe in the balanced capture probe library. In some embodiments, the desired sequencing depth profile is uniform. In some embodiments, the desired sequencing depth profile is non-uniform. In some embodiments, the desired sequencing depth profile is a sequencing depth above a predetermined minimum sequencing depth. In some embodiments, the subsequent amount is further selected to minimize a total number of sequencing reads.

The sequencing library can be enriched using a capture probe library by extending the capture probes that hybridize to the nucleic acid molecules, such as by direct capture sequencing. The nucleic acid molecules are used as a template for extension of the capture probe, thereby forming an extended capture probe with a sequence complementary to the nucleic acid molecule. The extended portion thereby includes bases that correspond to contiguous loci in the region of interest. Because the capture probe is extended using the nucleic acid molecule as a template, and the nucleic acid molecule can include bases both in and out of the region of interest, the extended portion can also include bases outside of the region of interest. The loci can be individual bases or can be contiguous sets of bases (e.g., 2 or more contiguous bases, 5 or more contiguous bases, 10 or contiguous more bases, 25 or more contiguous bases, 50 or more contiguous bases, or 100 or more contiguous bases). The extended capture probe can be amplified, for example by bridge amplification on a surface (e.g., a sequencing flow cell) before being sequenced.

Different capture probes in the capture probe library can be substantially complementary to different portions of the region of interest or portions adjacent to the region of interest, and can therefore hybridize to different nucleic acid molecules in the sequencing library. The region of interest need not be a contiguous sequence, and can optionally include two or more non-contiguous sub-regions. The region of interest can be divided into one or more tiles (which may or may not be contiguous). The tiles can be the same size or of different sizes, and each tile comprises one locus or a plurality of contiguous loci. In some embodiments, the tiles comprise 1 or more loci, about 5 or more loci, about 10 or more loci, about 25 or more loci, about 50 or more contiguous loci, about 75 or more contiguous loci, about 100 or more contiguous loci, about 200 or more contiguous loci, about 500 or more contiguous loci, or about 1000 or more contiguous loci. The capture probes can be assigned to the nearest tile for the purposes of determining a simulated sequencing depth, as explained in further detail below.

In some embodiments, one or more of the loci are incorporated into a single extended capture probe (that is, one or more of a particular locus in the region of interest may be incorporated into only a single capture probe). In some embodiments, one or more of the loci are incorporated into two or more probes. This can occur, for example, if a capture probe is extended to incorporate one or more loci beyond the start of extension of another capture probe in the capture probe library. This can also occur, for example, if two capture probes in the capture probe library hybridize to opposite strands of the region of interest and are extended toward each other such that one strand of one or more loci is incorporated into the first extended capture probe and the complementary strand of the same one or more loci is incorporated into the second extended capture probe.

The extended capture probes can be sequenced, and a sequencing depth (which can be normalized, for example by GC bias correct or mappability) determined at one or more contiguous loci within the region of interest. For one or more of the contiguous loci, a sequencing depth can be determined which is attributable to each capture probe in the capture probe library. A sequencing depth can also be determined which is attributable to the capture probe library (that is, the sum of the sequencing depths attributable to each of the capture probes in the capture probe library). In some embodiments, the sequencing depth attributable to one or more of the capture probes is assumed to be zero if the capture probe is substantially complementary to a portion of the region of interest distant from the locus (for example, if the capture probe is assignable to a different tile as the locus, or if the capture probe and the locus are separated by about 50 or more loci, about 75 or more loci, about 100 or more loci, about 200 or more loci, about 500 or more loci, or about 1000 or more loci).

The sequencing depth at any given locus is based on the number of sequencing reads that can be mapped to that locus after alignment with a reference sequence. In some embodiments, the sequencing depth is normalized, for example by GC bias correction or a correction for the mappability of the sequencing reads. In some embodiments, a plurality of sequencing libraries are sequenced, and the sequencing depth attributable to each capture probe in the capture probe library and the sequencing depth attributable to the capture probe library is an average sequencing depth or a minimum confidence sequencing depth. The average sequencing depth can be a median sequencing depth or a mean sequencing depth. The minimum confidence sequencing depth is the average sequencing depth minus a measure of variance in the distribution. The measure of variance can be, for example, one or more, two or more, or three or more standard deviations, or one or more, two or more, or three or more interquartile ranges. For example, in some embodiments the sequencing depth is a median minus two times the interquartile range of a plurality of sequencing library.

A balanced capture probe library can be constructed by combining at least a fraction of the capture probes at a subsequent known amount selected to obtain a desired sequencing depth profile at substantially all of the one or more contiguous loci attributable to the balanced capture probe library. The term “substantially all” is used to refer to the fact that noise and certain non-specific interactions may cause a small portion of the contiguous loci to have an attributable sequencing depth outside of the desired sequencing depth profile. In some embodiments, about 95% or more, about 96% or more, about 97% or more, about 98% or more, about 99% or more, about 99.5% or more, about 99.9% or more of the contiguous loci within the tile have a sequencing depth attributable to the balanced capture probe library within the desired sequencing depth profile.

The desired sequencing depth profile can be a sequencing depth above a predetermined minimum sequencing depth. For example, in some embodiments, the desired sequencing depth profile is a sequencing depth of 1 read or more, 2 reads or more, 5 reads or more, 10 reads or more, 15 reads or more, 20 reads or more, or 25 reads or more. The sequencing depth profile can also or alternatively be a sequencing depth below a maximum sequencing depth, such as about 1000 reads or fewer, about 500 reads or fewer, about 250 reads or fewer, 200 reads or few, 150 reads or fewer, or 100 reads or fewer.

The desired sequencing depth profile can be uniformly or non-uniformly applied to the plurality of loci within the tile. For example, in some embodiments, a non-uniformly applied sequencing depth profile comprises a first portion of the loci within the tile having first predetermined minimum sequencing depth and a second portion of the loci within the tile having a second predetermined minimum sequencing depth. Determining the subsequent amounts for the capture probes for a non-uniform sequencing depth profile across a plurality of tiles is facilitated by obtaining a simulated sequencing depth on a tile-by-tile basis.

The subsequent known amount of each capture probe is based on the initial known of amount of the capture probe, the sequencing depth at the one or more contiguous loci attributable to the capture probe, and the determined sequencing depth at the one or more contiguous loci attributable to the capture probe library.

In some embodiments, the initial known amount is an initial known volume, and the subsequent amount is a subsequent known volume. In some embodiments, the initial known amount is an initial known mass, and the subsequent known amount is a subsequent known mass. In some embodiments, the initial known amount is an initial known number of moles and the subsequent amount is a subsequent known number of moles.

In some embodiments, the subsequent known amount of each capture probe is selected by simulating a sequencing depth for at least one simulated capture probe amount at one or more of the contiguous loci. In some embodiments, the subsequent known amount of each capture probe is selected by simulating a sequencing depth for at least one simulated capture probe amount for two or more capture probes at one or more of the contiguous loci. Sequencing depth is approximately proportional to the amount of capture probe included in the capture probe library (with some amount of noise between samples and capture probes). Therefore, a sequencing depth for a capture probe can be simulated by changing the determined sequencing depth proportional to the change of a simulated amount from the initial known amount. That is:

$$\frac{{A}_{i , simulated}}{{A}_{i , known}} \approx \frac{{d}_{i , simulated}}{{d}_{i , known}}$$

wherein A_{i,known} is the known amount of capture probe i, A_{i,simulated} is the simulated amount of capture probe i, d_{i,known} is the determined sequencing depth attributable to capture probe i at the known amount, and d_{i,simulated} is the simulated sequencing depth attributable to capture probe i at the simulated amount.

Selecting the subsequent amount of each capture probe can include simulating a capture probe amount and a sequencing depth at one or more loci within a tile based on the simulated capture probe amount for one or more capture probes. This can be done iteratively to select finer amounts of the capture probe. One or more poor performing capture probes can be omitted from the simulation or from the construction of the balanced capture probe library (that is, the subsequent known for some of the capture probes can be zero). For example, in some embodiments, capture probes with particularly low (for example, first percentile or lower, or four or more interquartile ranges below the average) or particularly high (for example, 99th percentile or higher, or four or more interquartile ranges above the average) sequencing depth at the locus most proximal to the capture probe are omitted. In some embodiments, capture probes with fast (for example, 99th percentile or higher, or four or more interquartile ranges above the average) or slow (for example, first percentile or lower, or four or more interquartile ranges below the average) decay across the contiguous loci are omitted. The term “decay” refers to the decay constant of an exponential fit to the capture probe profile (such as those shown in FIGS. 2A-2I).

During the simulation of the sequencing depth at the one or more loci within the tile, a plurality of simulated amounts of each capture probe is selected based on the initial amount of the capture probe. For example, the simulated amount can range from 0.1-fold to 10-fold of the initial amount. In some embodiments, the simulation includes 3 or more, 4 or more, 5 or more, 6 or more, 7 or more, 8 or more, 9 or more, 10 or more, 12 or more, 15 or more, or 20 or more simulated amounts. Solely by way of example, the simulated amounts can be 0.2-fold, 0.4-fold, 0.8-fold, 1-fold, 2-fold, 4-fold, 6-fold, and 8-fold the initial amount. For each of the simulated amounts of the capture probes, a simulated sequencing depth at one or more contiguous loci can be determined by scaling the probe's coverage profile accordingly (e.g., a simulated amount of 2-fold for a given probe would double the coverage profile of the probe). Combinations of capture probes at the different simulated capture probe amounts can be used to determine a simulated sequencing depth attributable to the simulated capture probe library. The combination of simulated capture probe amounts for the capture probes that yield a summed sequencing depth (that is a sequencing depth attributable to the simulated library) in accordance with a desired sequencing depth profile (e.g., greater than the predetermined minimum sequencing depth) can then be selected. In some embodiments, the simulated capture probe amounts are further selected to obtain a sequencing depth below a maximum sequencing depth. For example, in some embodiments, the combination of simulated capture probe amounts that results in the lowest sequencing depth above the predetermined minimum sequencing depth is selected. In some embodiments, the simulated capture probe amounts are selected to obtain a sequencing depth below a predetermined maximum sequencing depth, which is set above the predetermined minimum sequencing depth. In some embodiments, the simulated capture probe amounts are selected to obtain the maximum sequencing depth below the predetermined maximum sequencing depth. In some embodiments, the combination of simulated capture probe amounts that consumes the fewest expected sequencing reads while still ensuring a lowest sequencing depth above the predetermined minimum sequencing depth is selected. This process can be performed iteratively with finer simulated sequencing probe amounts. Solely by way of example, if a simulated sequencing depth for a capture probe was selected as a 4-fold of the initial amount after sampling eight different simulated amounts ranging from 0.2-fold to 8-fold of the initial amount during the first iteration, the second iteration can include simulating capture probe amounts ranging from 2-fold to 6-fold (such as 2-fold, 2.5-fold, 3-fold, 4-fold, 4.5-fold, 5-fold, 5.5-fold, and 6-fold). This iterative process can continue for one or more, two or more, three or more, four or more, five or more, six or more, seven or more, or eight or more iterations.

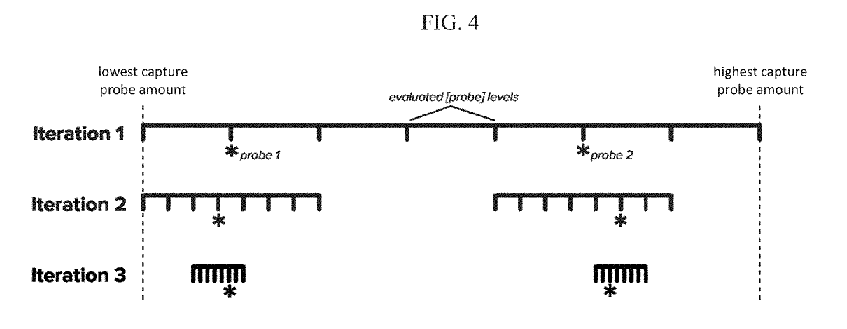

An example process of simulating capture probe amounts and sequencing depth is illustrated in FIG. 4. In the illustrated example, a sequencing depth at a locus is attributed to two capture probes (probe 1 and probe 2), although at some loci a sequencing depth may be attributed to one, two, three, four, five, six seven, eight, nine, ten or more capture probes. In the embodiment illustrated in FIG. 4, a sequencing depth is simulated for each of probe 1 and probe 2 at eight different simulated capture probe concentrations. Although eight different simulated capture probe concentrations are used in the embodiments illustrated in FIG. 4, any of 2, 3, 4, 5, 6, 7, 8, 9, 10 or more, 12 or more, 15 or more, or 20 or more simulated amounts can be used. The combination of simulated sequencing depths for each of the various simulated capture probe amounts (64 combinations in the illustrated example) is summed and compared to the desired sequencing depth profile (e.g., predetermined minimum sequencing depth). The combination of simulated capture probe amounts for the capture probes that yield a summed sequencing depth in accordance with the desired sequencing depth profile (e.g., greater than the predetermined minimum sequencing depth) can then be selected. One or more additional selection criteria can be used to select the combination of simulated capture probe amounts that would be in accordance with the desired sequencing depth profile when two or more combinations of simulated capture probes amounts would be in accordance. For example, in some embodiments, the combination of simulated capture probe amounts that would result in the fewest sequencing reads above a predetermined minimum sequencing depth is selected. In the example illustrated in FIG. 4, once the simulated subsequent amounts for capture probe 1 and capture probe 2 are selected, a second round of selection can occur (i.e., Iteration 2) with a narrower window of simulated capture probe amounts. For example, simulated sequencing depths attributable to capture probe 1 or capture probe 2 can be determined using simulated capture probe amounts based on the previously selected simulated capture probe amounts. In the illustrated example, eight capture probe amounts are simulated, which are selected from within the window of simulated capture probe amounts form the previous iteration (i.e., from with simulated capture probe amounts one step up and one step down from the selected simulated capture probe amount in the previous selection round). Although eight different simulated capture probe concentrations are used in Iteration 2 of the embodiment illustrated in FIG. 4, any of 2, 3, 4, 5, 6, 7, 8, 9, 10 or more, 12 or more, 15 or more, or 20 or more simulated concentrations can be used. The combination of simulated sequencing depths for each of the various simulated capture probe amounts (64 combinations in the illustrated example) in Iteration 2 are summed and compared to the desired sequencing depth profile. The combination of simulated capture probe amounts for the capture probes that yield a summed sequencing depth in accordance with the desired sequencing depth profile (e.g., greater than the predetermined minimum sequencing depth) can then be selected. In the example illustrated in FIG. 4, once the simulated subsequent amounts for capture probe 1 and capture probe 2 are selected, a third round of selection can occur (i.e., Iteration 3) with a further narrower window of simulated capture probe amounts. For example, simulated sequencing depths attributable to capture probe 1 or capture probe 2 can be determined using simulated capture probe amounts based on the selected simulated capture probe amounts from Iteration 2. In the illustrated example, eight capture probe amounts are simulated, which are selected from within the window of simulated capture probe amounts form the previous iteration (i.e., from with simulated capture probe amounts one step up and one step down from the selected simulated capture probe amount in the previous selection round). Although eight different simulated capture probe concentrations are used in Iteration 3 of the embodiment illustrated in FIG. 4, any of 2, 3, 4, 5, 6, 7, 8, 9, 10 or more, 12 or more, 15 or more, or 20 or more simulated concentrations can be used. The combination of simulated sequencing depths for each of the various simulated capture probe amounts (64 combinations in the illustrated example) in Iteration 2 are summed and compared to the desired sequencing depth profile. The combination of simulated capture probe amounts for the capture probes that yield a summed sequencing depth in accordance with the desired sequencing depth profile (e.g., greater than the predetermined minimum sequencing depth) can then be selected. Although the simulation illustrated in FIG. 4 shows three iteration of simulating capture probe amounts and sequencing depths, in some embodiments only a single round of simulation is performed. In some embodiments, two or more, three or more, four or more, five or more, six or more, seven or more, or eight or more iterations of simulating capture probe amounts is performed. The simulated capture probe amount in the final iteration can be used as the subsequent known amount of the capture probe when constructing the second (or balanced) capture probe library.

Any other method of optimizing a set of parameter values to satisfy multiple quantitative restrains (such as simulated capture probe amounts and simulated sequencing depth) can be used. Exemplary methods include simulated annealing or the Nelder-Mead method. For example, in some embodiments, a predetermined minimum sequencing depth is defined as a constraint; the coverage profile of one or more sequencing probes is defined as a function of the amount of all capture probes in a tile or a tile supergroup, which may be bounded by a predetermined maximum amount of the capture probe and/or a predetermined minimum amount of the capture probe, and the function to be minimized is the number of sequencing reads subject to the predetermined minimum sequencing depth constraint and the function of the capture probes.

Simulations can be performed on a tile-by-tile basis within the region of interest. That is, a simulation can be performed for each of the tiles within the region of interest. The tiles can be the same length or of different lengths. For example, the region of interest can be divided into sub-regions, such as exons, wherein each exon is a tile. Some exons are long and can be divided into a plurality of contiguous tiles. Occasionally, one or more of the capture probe assigned to a given tile can have a capture probe profile that extends into (and contributes to the sequencing depth) of one or more loci in another (generally adjacent) tile. This can occur, for example, when an exon is divided into two or more tiles and a capture probe enriches a portion of an adjacent tile. In some embodiments, the tiles with overlapping capture probe profiles are merged to form a single larger tile. In some embodiments, sequencing depth from a capture probe assigned to a different tile is ignored. The desired sequencing depth profile for the one or more tiles can be uniform or non-uniform. For example, in some embodiments, a first tile can have a first desired sequencing depth profile and the second tile can have a second desired sequencing depth profile.

In some embodiments, simulation is performed on a tile supergroup, which may include two or more (such as 3, 4, or 5 or more) tiles. The tiles in the tile supergroup may be contiguous or non-contiguous. For example, a capture probe may contribute to a sequencing depth of two or more non-contiguous tiles, and the non-contiguous tiles may be grouped together in a single tile supergroup for simulation.

The balanced capture probe library is constructed by combining at least a fraction (or all) of the capture probes at the subsequent known amounts of each capture probe in the balanced capture probe library. Not all capture probes included in the first capture probe library need be included in the balanced capture probe library. For example, in some embodiments, constructing the balanced capture probe library by combining at least a fraction of the capture probes comprises omitting (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold. The desired sequencing depth profile can be, for example, a predetermined minimum sequencing depth at each of the one or more contiguous loci.

The method of preparing the balance capture probe library can be performed iteratively. For example, the method can further comprise enriching the sequencing library using the balanced capture probe library; sequencing the sequencing library enriched using the balanced capture probe library; determining at each of the one or more loci within the tile: (i) a sequencing depth attributable to each capture probe in the balanced capture probe library, and (ii) a sequencing depth attributable to the balanced capture probe library; selecting a second subsequent known amount of each capture probe based on the subsequent known amount of said capture probe, the sequencing depth at each locus attributable to said capture probe, and the sequencing depth at each locus attributable to the balanced capture probe library, wherein the second subsequent amount is selected to obtain a desired sequencing depth profile at substantially all of the contiguous loci; and constructing a re-balanced capture probe library by combining at least a fraction of the capture probes at the second subsequent known amount of each capture probe in the re-balanced capture probe library. In some embodiments, the second subsequent amount is further selected to minimize a total number of sequencing reads. This method can be iteratively repeated by rebalancing the re-balanced (i.e., third) capture probe library (or any later constructed capture probe library) one or more, two or more, three or more, four or more, or five or more times.

The balanced capture probe library can be used to enrich a test sequencing library. For example, in some embodiments a method of enriching a test sequencing library comprises (1) combining the test sequencing library comprising test nucleic acid molecules with the capture probe library prepared by sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining at each locus within a tile of the region of interest comprising one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the one or more contiguous loci within the tile; and constructing the balanced capture probe library by combining at least a fraction of the capture probes at the subsequent known amount of each capture probe in the balanced capture probe library; and (2) extending the capture probes that hybridize to the nucleic acid molecules. In some embodiments a method of sequencing the test sequencing library comprises enriching the test sequencing library with the balanced capture probe library and sequencing the enriched test sequencing library. In some embodiments, the method further comprises amplifying (for example, by bridge amplification) the enriched test sequencing library.

Two or more balanced (including rebalanced) capture probe libraries can be combined to form a pooled capture probe library. Two or more balanced (including rebalanced) capture probe libraries can be combined to form a pooled capture probe library.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the one or more contiguous loci within the tile; and constructing the balanced capture probe library by combining a at least a fraction the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain predetermined minimum sequencing depth at substantially all of the one or more contiguous loci within the tile; and constructing the balanced capture probe library by combining a at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain predetermined minimum sequencing depth and remain below a predetermined maximum sequencing depth at substantially all of the one or more contiguous loci within the tile; and constructing the balanced capture probe library by combining a at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a sequencing library comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing library, and wherein an initial known amount of each capture probe is used to form the first capture probe library; sequencing the enriched sequencing library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a non-uniform desired sequencing depth profile at substantially all of the one or more contiguous loci within the tile; and constructing the balanced capture probe library by combining at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a sequencing library comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing library, and wherein an initial known amount of each capture probe is used to form the first capture probe library; sequencing the enriched sequencing library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the one or more contiguous loci within the tile; and constructing the balanced capture probe library by combining at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a sequencing library comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing library, and wherein an initial known amount of each capture probe is used to form the first capture probe library; sequencing the enriched sequencing library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain predetermined minimum sequencing depth at substantially all of the one or more contiguous loci within the tile; and constructing the balanced capture probe library by combining at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a sequencing library comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing library, and wherein an initial known amount of each capture probe is used to form the first capture probe library; sequencing the enriched sequencing library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain predetermined minimum sequencing depth and remain below a predetermined maximum sequencing depth at substantially all of the one or more contiguous loci within the tile; and constructing the balanced capture probe library by combining at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a sequencing library comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing library, and wherein an initial known amount of each capture probe is used to form the first capture probe library; sequencing the enriched sequencing library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a non-uniform desired sequencing depth profile at substantially all of the one or more contiguous loci within the tile; and constructing the balanced capture probe library by combining at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a sequencing library comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing library, and wherein an initial known amount of each capture probe is used to form the first capture probe library; sequencing the enriched sequencing library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a non-uniform desired sequencing depth profile at substantially all of the one or more contiguous loci within the tile by simulating a sequencing depth for at least one simulated capture probe amount; and constructing the balanced capture probe library by combining at least a fraction the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a f of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a sequencing library comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing library, and wherein an initial known amount of each capture probe is used to form the first capture probe library; sequencing the enriched sequencing library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the one or more contiguous loci within the tile by simulating a sequencing depth for at least one simulated capture probe amount; and constructing the balanced capture probe library by combining at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a sequencing library comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing library, and wherein an initial known amount of each capture probe is used to form the first capture probe library; sequencing the enriched sequencing library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain predetermined minimum sequencing depth at substantially all of the one or more contiguous loci within the tile by simulating a sequencing depth for at least one simulated capture probe amount; and constructing the balanced capture probe library by combining at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a sequencing library comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing library, and wherein an initial known amount of each capture probe is used to form the first capture probe library; sequencing the enriched sequencing library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe by simulating a sequencing depth for at least one simulated capture probe amount, the sequencing depth at each locus attributable to said capture probe, and the sequencing depth at each locus attributable to the first capture probe library, wherein the subsequent amount is selected to obtain predetermined minimum sequencing depth and remain below a predetermined maximum sequencing depth at substantially all of the one or more contiguous loci within the tile; and constructing the balanced capture probe library by combining at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a sequencing library comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing library, and wherein an initial known amount of each capture probe is used to form the first capture probe library; sequencing the enriched sequencing library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe by simulating a sequencing depth for at least one simulated capture probe amount, the sequencing depth at each locus attributable to said capture probe, and the sequencing depth at each locus attributable to the first capture probe library, wherein the subsequent amount is selected to obtain a non-uniform desired sequencing depth profile at substantially all of the one or more contiguous loci within the tile; and constructing the balanced capture probe library by combining at least a fraction of the capture probes at the subsequent known amount of each capture probe in the balanced capture probe library. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a plurality of sequencing libraries comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing libraries, and wherein an initial known amount of each capture probe is used to form the first capture probe library; sequencing the plurality of enriched sequencing libraries; determining at each locus within a tile of the region of interest comprising a one or more contiguous loci: (i) a minimum confidence sequencing depth attributable to each capture probe in the first capture probe library, and (ii) a minimum confidence sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a non-uniform desired sequencing depth profile at substantially all of the one or more contiguous loci within the tile by simulating a sequencing depth for at least one simulated capture probe amount; and constructing the balanced capture probe library by combining at least a fraction of the capture probes at the subsequent known amount of each capture probe in the balanced capture probe library. In some embodiments, the minimum confidence sequencing depth is a median sequencing depth minus two times an interquartile range determined from a distribution of sequencing depths at each locus from the plurality of sequencing libraries. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a plurality of sequencing libraries comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing libraries, and wherein an initial known amount of each capture probe is used to form the first capture probe library; sequencing the plurality of enriched sequencing libraries; determining at each locus within a tile of the region of interest comprising a one or more contiguous loci: (i) a minimum confidence sequencing depth attributable to each capture probe in the first capture probe library, and (ii) a minimum confidence sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the one or more contiguous loci within the tile by simulating a sequencing depth for at least one simulated capture probe amount; and constructing the balanced capture probe library by combining at least a fraction of the capture probes at the subsequent known amount of each capture probe in the balanced capture probe library. In some embodiments, the minimum confidence sequencing depth is a median sequencing depth minus two times an interquartile range determined from a distribution of sequencing depths at each locus from the plurality of sequencing libraries. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a plurality of sequencing libraries comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing libraries, and wherein an initial known amount of each capture probe is used to form the first capture probe library; sequencing the plurality of enriched sequencing libraries; determining at each locus within a tile of the region of interest comprising a one or more contiguous loci: (i) a minimum confidence sequencing depth attributable to each capture probe in the first capture probe library, and (ii) a minimum confidence sequencing depth attributable to the first capture probe library; selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain predetermined minimum sequencing depth at substantially all of the one or more contiguous loci within the tile by simulating a sequencing depth for at least one simulated capture probe amount; and constructing the balanced capture probe library by combining at least a fraction of the capture probes at the subsequent known amount of each capture probe in the balanced capture probe library. In some embodiments, the minimum confidence sequencing depth is a median sequencing depth minus two times an interquartile range determined from a distribution of sequencing depths at each locus from the plurality of sequencing libraries. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a plurality of sequencing libraries comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing libraries, and wherein an initial known amount of each capture probe is used to form the first capture probe library; sequencing the plurality of enriched sequencing libraries; determining at each locus within a tile of the region of interest comprising a one or more contiguous loci: (i) a minimum confidence sequencing depth attributable to each capture probe in the first capture probe library, and (ii) a minimum confidence sequencing depth attributable to the first capture probe library; selecting a subsequent known amount of each capture probe based on the initial known amount of said capture probe by simulating a sequencing depth for at least one simulated capture probe amount, the sequencing depth at each locus attributable to said capture probe, and the sequencing depth at each locus attributable to the first capture probe library, wherein the subsequent amount is selected to obtain predetermined minimum sequencing depth and remain below a predetermined maximum sequencing depth at substantially all of the one or more contiguous loci within the tile; and constructing the balanced capture probe library by combining at least a fraction of the capture probes at the subsequent known amount of each capture probe in the balanced capture probe library. In some embodiments, the minimum confidence sequencing depth is a median sequencing depth minus two times an interquartile range determined from a distribution of sequencing depths at each locus from the plurality of sequencing libraries. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

In some embodiments, there is a method of preparing a balanced capture probe library comprising enriching a plurality of sequencing libraries comprising a plurality of nucleic acid molecules using a first capture probe library by extending the capture probes that hybridize to the nucleic acid molecules, wherein each capture probe comprises a sequence that is substantially complementary to a portion of or adjacent to a region of interest included in the sequencing libraries, and wherein an initial known amount of each capture probe is used to form the first capture probe library; sequencing the plurality of enriched sequencing libraries; determining at each locus within a tile of the region of interest comprising a one or more contiguous loci: (i) a minimum confidence sequencing depth attributable to each capture probe in the first capture probe library, and (ii) a minimum confidence sequencing depth attributable to the first capture probe library; determining at each locus within a tile of the region of interest comprising a one or more contiguous loci: (i) a sequencing depth attributable to each capture probe in the first capture probe library, and (ii) a sequencing depth attributable to the first capture probe library; selecting a subsequent known amount of each capture probe based on the initial known amount of said capture probe by simulating a sequencing depth for at least one simulated capture probe amount, the sequencing depth at each locus attributable to said capture probe, and the sequencing depth at each locus attributable to the first capture probe library, wherein the subsequent amount is selected to obtain a non-uniform desired sequencing depth profile at substantially all of the one or more contiguous loci within the tile; and constructing the balanced capture probe library by combining at least a fraction of the capture probes at the subsequent known amount of each capture probe in the balanced capture probe library. In some embodiments, the minimum confidence sequencing depth is a median sequencing depth minus two times an interquartile range determined from a distribution of sequencing depths at each locus from the plurality of sequencing libraries. In some embodiments, the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles. In some embodiments, the desired sequencing depth profile is uniform for each of the tiles in the plurality of tiles. In some embodiments, the desired sequencing depth profile is non-uniform for each of the tiles in the plurality of tiles. In some embodiments, the initial known amount is an initial known volume, and the subsequent known amount is a subsequent known volume. In some embodiments, the initial known amount is an initial known mass or an initial known number of moles, and the subsequent known amount is a subsequent known mass or a subsequent known number of moles. In some embodiments, the initial known amount is an initial known relative amount and the subsequent known amount is a subsequent known relative amount. In some embodiments, the sequencing depth attributable to the capture probe library for one or more loci is attributable only to a single capture probe. In some embodiments, the sequencing depth attributable to the capture probe library for one or more loci is attributable to two or more capture probes. In some embodiments, two or more of the capture probes in the capture probe library bind to opposite nucleic acid strands. In some embodiments, constructing the balanced capture probe library by combining at least a fraction of the capture probes comprises omitting: (i) one or more capture probes with a decay coefficient above a maximum predetermined threshold or below a minimum predetermined threshold; (ii) one or more capture probes that results in a number of sequencing reads at one or more contiguous loci above a maximum predetermined threshold or below a minimum predetermined threshold; or (iii) one or more capture probes with a variation above a predetermined threshold.

### EXEMPLARY EMBODIMENTS

### Embodiment 1

A method of preparing a balanced capture probe library comprising:
sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library;
determining a sequencing depth attributable to each capture probe;
selecting a subsequent known amount of each capture probe based on the initial known amount and the sequencing depth attributable to said capture probe, wherein the subsequent known amount of each capture probe is selected to minimize a difference between a predicted sequencing depth profile and a desired sequencing depth profile, and wherein minimization of the difference is constrained by a minimum subsequent amount or a maximum subsequent amount of each capture probe; and
constructing the balanced capture probe library by combining at least a fraction of the capture probes at the subsequent known amount of each capture probe in the balanced capture probe library.

### Embodiment 2

The method of embodiment 1, wherein the desired sequencing depth profile is non-uniform.

### Embodiment 3

A method of preparing a balanced capture probe library comprising:
sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library;
determining a sequencing depth attributable to each capture probe;
selecting a subsequent known amount of each capture probe based on the initial known amount and the sequencing depth attributable to said capture probe, wherein the subsequent known amount of each capture probe is selected to minimize a difference between a predicted sequencing depth profile and a non-uniform desired sequencing depth profile; and
constructing the balanced capture probe library by combining at least a fraction of the capture probes at the subsequent known amount for each capture probe in the balanced capture probe library.

### Embodiment 4

The method of any one of embodiments 1-3, wherein the difference between the predicted sequencing depth profile and the desired sequencing depth profile is defined by an objective function.

### Embodiment 5

The method of embodiment 4, wherein the desired sequencing depth profile is implied by the objective function.

### Embodiment 6

The method of any one of embodiments 1-5, wherein the initial known amount is an initial known volume and the subsequent known amount is a subsequent known volume.

### Embodiment 7

The method of any one of embodiments 1-6, wherein the initial known amount is an initial known mass or an initial known number of moles, and the subsequent known amount is a subsequent known mass or a subsequent known number of moles.

### Embodiment 8

The method of any one of embodiments 1-7, wherein the initial known amount is an initial known relative amount and the subsequent known amount is a subsequent known relative amount.

### Embodiment 9

The method of any one of embodiments 1-8, wherein the sequencing depth is based on a number of sequencing reads that comprise a sequence substantially complementary to a sequence within the capture probe.

### Embodiment 10

The method of any one of embodiments 1-9, wherein the sequencing depth is based on a number of sequencing reads that comprise a sequence substantially complementary to at least half of the capture probe.

### Embodiment 11

The method of embodiment 9 or 10, wherein the number of sequencing reads is a number of consensus sequencing reads.

### Embodiment 12

The method of any one of embodiments 9-11, wherein the number of sequencing reads is a number of duplex consensuses sequencing reads.

### Embodiment 13

The method of any one of embodiments 1-12, wherein the capture probes are not substantially complementary to overlapping portions of the region of interest.

### Embodiment 14

The method of any one of embodiments 1-13, wherein at least two capture probes in the plurality of capture probes are substantially complementary to overlapping portions of the region of interest.

### Embodiment 15

The method of any one of embodiments 1-14, wherein the method comprises obtaining for each capture probe a binding fraction based on the sequencing depth attributable to the capture probe; and wherein the subsequent known amount is based on the initial known amount and the binding fraction.

### Embodiment 16

The method of any one of embodiments 1-15, wherein the method comprises obtaining for each capture probe a binding fraction, wherein the binding fraction is determined for the fraction of nucleic acid molecules comprising a segment substantially complementary to at least half of the capture probe that bound to the capture probe during enrichment of the sequencing library based on the sequencing depth attributable to the capture probe; and wherein the subsequent known amount is based on the initial known amount and the fraction.

### Embodiment 17

The method of embodiment 15 or 16, wherein obtaining the binding fraction comprises approximating the sequencing depth for a given capture probe i as a Poisson distribution:

dᵢₛ=Poisson(Nₛπᵢ)
wherein:
   dᵢₛ is the determined sequencing depth attributable to the capture probe i;
   Nₛ is the number of nucleic acid molecules in sequencing library s; and
   πᵢ is the binding fraction for capture probe i.

### Embodiment 18

The method of embodiment 17, wherein πᵢ is determined by fitting a maximum likelihood model or a Markov chain Monte Carlo model.

### Embodiment 19

The method of any one of embodiments 1-18, wherein the difference is defined by an objective function of the subsequent amounts of the capture probes in the balanced capture probe library, according to:

$$f ( \vec{v} ) = V ( \vec{π} ( \vec{v} ) )$$

$$wherein : {( \vec{π} ( \vec{v} ) )}_{i} = {( 1 + \frac{{γ}_{i}}{{v}_{i} {c}_{i}} )}^{- 1}$$

wherein:
   V( ) is a user-defined objective function for the capture probe library;
   (\vec{π}(\vec{v}))ᵢ is the binding fraction for capture probe i for the vector \vec{π}(\vec{v});
   cᵢ is an effective concentration for capture probe i for the vector \vec{π}(\vec{v});
   vᵢ is the subsequent amount of capture probe i for the vector \vec{π}(\vec{v}); and
   γᵢ is the initial amount of capture probe i for the vector \vec{π}(\vec{v}).

### Embodiment 20

The method of any one of embodiments 1-19, wherein the difference is an objective function defined by a coefficient of variation.

### Embodiment 21

The method of any one of embodiments 1-20, wherein the plurality of capture probes comprises 10 or more unique capture probes.

### Embodiment 22

The method of any one of embodiments 1-21, wherein the capture probes are about 20 to about 160 bases in length.

### Embodiment 23

The method of any one of embodiments 1-22, wherein the plurality of capture probes comprise DNA capture probes.

### Embodiment 24

The method of any one of embodiments 1-23, wherein the plurality of capture probes comprise RNA capture probes.

### Embodiment 25

The method of any one of embodiments 1-24, wherein the capture probes are biotinylated.

### Embodiment 26

The method of any one of embodiments 1-25, further comprising:
enriching the sequencing library using the balanced capture probe library;
sequencing the sequencing library enriched using the balanced capture probe library; determining a sequencing depth attributable to each capture probe in the balanced capture probe library;
selecting a second subsequent known amount of each capture probe based on the subsequent known amount of said capture probe in the balanced capture probe library and the sequencing depth attributable to said capture probe, wherein the second subsequent known amount of each capture probe is selected to minimize a difference between a second predicted sequencing depth profile and the desired sequencing depth profile; and
constructing a re-balanced capture probe library by combining at least a fraction of the capture probes at the second subsequent known amount of each capture probe in the re-balanced capture probe library.

### Embodiment 27

The method of embodiment 26, wherein minimization of the difference is constrained by a minimum second subsequent amount or a maximum second subsequent amount of each capture probe.

### Embodiment 28

The method of any one of embodiments 1-27, wherein constructing the balanced capture probe library comprises combining each capture probe in the first capture probe library at the subsequent known amount of each capture probe in the balanced capture probe library.

### Embodiment 29

The method of any one of embodiments 1-27, wherein the sequencing library is an RNA sequencing library or a DNA sequencing library.

### Embodiment 30

A balanced capture probe library made according to the method of any one of embodiments 1-29.

### Embodiment 31

A method of enriching a test sequencing library comprising:
combining the test sequencing library comprising nucleic acid molecules with the capture probe library prepared according to the method of any one of embodiments 1-28; and
selecting nucleic acid molecules from the test sequencing library that hybridize with the capture probes in the capture probe library.

### Embodiment 32

A method of sequencing a test sequencing library comprising:
enriching the test sequencing library according to the method of embodiment 31; and
sequencing the enriched test sequencing library.

### Embodiment 33

The method of embodiment 31 or 32, further comprising amplifying the enriched test sequencing library.

### Embodiment 34

The method of any one of embodiments 31-33, further comprising removing the capture probes from the enriched test sequencing library.

### Embodiment 35

The method of any one of embodiments 31-34, wherein the test sequencing library comprises cell-free DNA.

### Embodiment 36

The method of embodiment 35, wherein the cell-free DNA comprises fetal cell-free DNA.

### Embodiment 37

The method of embodiment 35, wherein the cell-free DNA comprises circulating tumor cell-free DNA.

### Embodiment 38

The method of any one of embodiments 31-34, wherein the test sequencing library comprises fragmented DNA derived from cells contained with a sample.

### Embodiment 39

The method of any one of embodiments 31-34, wherein the test sequencing library is an RNA sequencing library.

### Embodiment 40

The method of any one of embodiments 31-39, wherein the nucleic acid molecules in the test sequencing library have an average length of about 100 bases to about 500 bases.

### Embodiment 41

The method of any one of embodiments 30-40, wherein the nucleic acid molecules in the test sequencing library are ligated to sequencing adapters comprising molecular barcodes.

### Embodiment 42

The method of any one of embodiments 1-41, wherein enriching the sequencing library comprises separating nucleic acid molecules in the sequencing library that are hybridized to the capture probes from nucleic acid molecules in the sequencing library that are not hybridized to the capture probes.

### Embodiment 43

A method of forming a pooled capture probe library comprising combing two or more balanced capture probe libraries prepared according to the method of any one of embodiments 1-29.

### Embodiment 44

A method of preparing a balanced capture probe library comprising:
sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library;
determining at each locus within a tile of the region of interest comprising a one or more contiguous loci:
   i. a sequencing depth attributable to each capture probe in the first capture probe library, and
   ii. a sequencing depth attributable to the first capture probe library;
   selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the one or more contiguous loci within the tile; and
constructing the balanced capture probe library by combining at least a fraction of the capture probes at the subsequent known amount of each capture probe in the balanced capture probe library.

### Embodiment 45

The method of embodiment 44, wherein the desired sequencing depth profile is a predetermined minimum sequencing depth at each of the one or more contiguous loci.

### Embodiment 46

The method of embodiment 44 or 45, wherein the subsequent amount is further selected to obtain a sequencing depth below a maximum sequencing depth.

### Embodiment 47

The method of any one of embodiments 44-46, wherein the tile comprises a plurality of loci and the desired sequencing depth profile is uniform for the plurality of loci within the tile.

### Embodiment 48

The method of any one of embodiments 44-46, wherein the tile comprises a plurality of loci and the desired sequencing depth profile is non-uniform for the plurality of loci within the tile.

### Embodiment 49

The method of any one of embodiments 44-48, further comprising enriching the sequencing library using the first capture probe library by extending the capture probes that hybridize to the nucleic acid molecules.

### Embodiment 50

The method of any one of embodiments 44-49, wherein the region of interest is divided into a plurality of tiles, each tile comprising a plurality of contiguous loci, and a sequencing depth attributable to each capture probe in the first capture probe library and a sequencing depth attributable to the first capture probe library is determined for each locus within the plurality of tiles; and wherein the subsequent amount of each capture probe is selected to obtain a desired sequencing depth profile at substantially all of the one or more contiguous loci within the plurality of tiles.

### Embodiment 51

The method of embodiment 50, wherein the desired sequencing depth profile is uniform for each of the tiles in the plurality of tiles.

### Embodiment 52

The method of embodiment 50, wherein the desired sequencing depth profile is non-uniform for each of the tiles in the plurality of tiles.

### Embodiment 53

The method of any one of embodiments 44-52, wherein the initial known amount is an initial known volume, and the subsequent known amount is a subsequent known volume.

### Embodiment 54

The method of any one of embodiments 44-52, wherein the initial known amount is an initial known mass or an initial known number of moles, and the subsequent known amount is a subsequent known mass or a subsequent known number of moles.

### Embodiment 55

The method of any one of embodiments 44-52, wherein the initial known amount is an initial known relative amount and the subsequent known amount is a subsequent known relative amount.

### Embodiment 56

The method of any one of embodiments 44-55, wherein the sequencing depth attributable to the capture probe library for one or more loci is attributable only to a single capture probe.

### Embodiment 57

The method of any one of embodiments 44-55, wherein the sequencing depth attributable to the capture probe library for one or more loci is attributable to two or more capture probes.

### Embodiment 58

The method of any one of embodiments 44-57, wherein two or more of the capture probes in the capture probe library bind to opposite nucleic acid strands.

### Embodiment 59

The method of any one of embodiments 44-58, wherein selecting the subsequent known amount of each capture probe comprises simulating a sequencing depth for at least one simulated capture probe amount.

### Embodiment 60

The method of any one of embodiments 44-59, wherein selecting the subsequent known amount of each capture probe comprises combining a simulated sequencing depth determined for two or more capture probes.

### Embodiment 61

The method of embodiment 59 or 60, wherein the simulated sequencing depth is simulated using a proportional relationship between the amount of the capture probe and the sequencing depth at the locus.

### Embodiment 62

The method of any one of embodiments 44-61, wherein:
the method comprises sequencing a plurality of sequencing libraries; and
the sequencing depth at each locus is a minimum confidence sequencing depth.

### Embodiment 63

The method of embodiment 62, wherein the minimum confidence sequencing depth is a median sequencing depth minus two times an interquartile range determined from a distribution of sequencing depths at each locus from the plurality of sequencing libraries.

### Embodiment 64

The method of any one of embodiments 44-63, wherein constructing the balanced capture probe library by combining at least a fraction of the capture probes comprises omitting:
i. one or more capture probes with a decay coefficient above a maximum predetermined threshold or below a minimum predetermined threshold;
ii. one or more capture probes that results in a number of sequencing reads at one or more contiguous loci above a maximum predetermined threshold or below a minimum predetermined threshold; or
iii. one or more capture probes with a variation above a predetermined threshold.

### Embodiment 65

The method of any one of embodiments 44-64, further comprising:
enriching the sequencing library using the balanced capture probe library;
sequencing the sequencing library enriched using the balanced capture probe library; determining at each locus within the tile:
i. a sequencing depth attributable to each capture probe in the balanced capture probe library, and
ii. a sequencing depth attributable to the balanced capture probe library;
selecting a second subsequent known amount of each capture probe based on the subsequent known amount of said capture probe, the sequencing depth at each locus attributable to said capture probe, and the sequencing depth at each locus attributable to the balanced capture probe library, wherein the second subsequent amount is selected to obtain a desired sequencing depth profile at substantially all of the one or more contiguous loci within the tile; and
constructing a re-balanced capture probe library by combining at least a fraction of the capture probes at the second subsequent known amount of each capture probe in the re-balanced capture probe library.

### Embodiment 66

The method of any one of embodiments 44-65, wherein the capture probes are about 20 to about 60 bases in length.

### Embodiment 67

A balanced capture probe library made according to any one of embodiments 44-66.

### Embodiment 68

A method of enriching a test sequencing library comprising:
combining the test sequencing library comprising test nucleic acid molecules with the capture probe library prepared according to the method of any one of embodiments 44-66; and
extending the capture probes that hybridize to the test nucleic acid molecules.

### Embodiment 69

A method of sequencing a test sequencing library comprising:
enriching the test sequencing library according to the method of embodiment 68; and
sequencing the enriched test sequencing library.

### Embodiment 70

The method of embodiment 68 or 69, further comprising amplifying the enriched test sequencing library.

### Embodiment 71

The method of any one of embodiments 68-70, wherein the test sequencing library comprises fragmented DNA derived from cells in a sample.

### Embodiment 72

The method of any one of embodiments 68-71 wherein the test sequencing library comprises cell-free DNA.

### Embodiment 73

A method of forming a pooled capture probe library comprising combining two or more balanced capture probe libraries made according to the method of any one of embodiments 44-66.

### EXAMPLES

### Example 1: Balanced Capture Probe Library with Minimized Variability

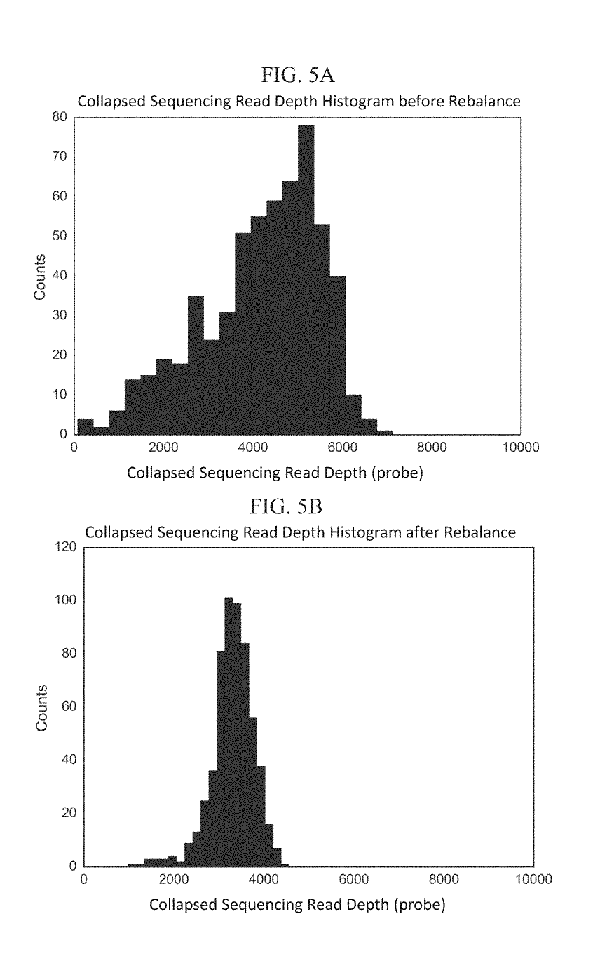

A capture probe library having 583 biotinylated DNA capture probes of 120 bases in length was formed by combining 2.0 uL of each individual capture probe at approximately equimolar concentrations. The capture probe library was then combined with a healthy cell-free DNA sequencing library comprising nucleic acid molecules bound to sequencing adapters (including molecular barcodes). Nucleic acid molecules in the sequencing library that hybridized to the capture probes in the capture probe library were isolated from the non-hybridized nucleic acid molecules, thereby forming the enriched sequencing library. The enriched sequencing library was PCR amplified and sequenced using an Illumina HiSeq2500 sequencer. Consensus sequences were formed by collapsing all sequencing reads associated with the same molecular barcodes. The sequencing reads were then aligned to the hg19 human genome and the sequencing depth attributable to each capture probe was determined by counting the number of consensus molecules observed that contained at least half of the sequence of the capture probe. The sequencing depth attributable to the capture probes is plotted as a histogram in FIG. 5A, where histogram measures the number of collapsed sequencing reads for each probe with at least 50% overlap with the probe of interest.

The capture probe library was balanced to minimize the difference between a predicted sequencing depth profile and a uniform desired sequencing depth profile. Minimum capture probe volumes were limited to 0.75 uL and maximum capture probe volumes were limited to 4 uL. To balance the capture probe library, for each capture probe a binding fraction of the nucleic acid molecules comprising a segment substantially complementary to at least half of the capture probe that bound to the capture probe during enrichment of the sequencing libraries was obtained by approximating the sequencing depth for each capture probe as a Poisson distribution and fitting a maximum likelihood model for the binding fraction. The coefficient of variation (standard deviation divided by the mean) of the predicted sequencing depths was minimized, subject to the aforementioned volume constraints, to determine subsequent volumes for each capture probe. A balanced capture probe library was constructed using the subsequent volumes for each capture probe. The balanced capture probe library was used to enrich the sequencing library, and the sequencing depths attributable to the capture probes in the balanced capture probe library are shown in FIG. 5B.

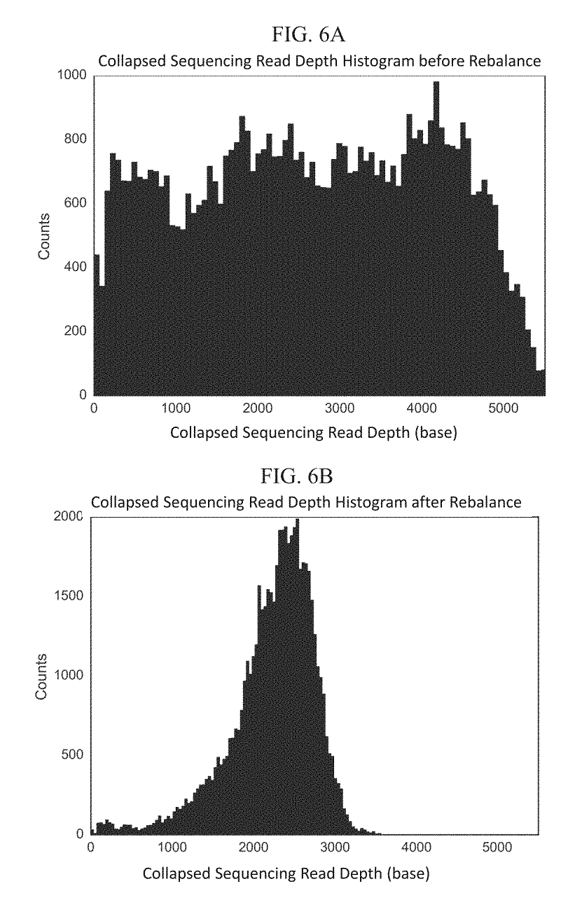

As can be seen in FIG. 5A and FIG. 5B, the rebalanced capture probe library results in a narrower distribution of probe counts. Before balancing, the sequencing depth distribution ranged from 0 to about 7000. After rebalancing, the sequencing depth peaked at 3500, and the number of counts with a sequencing depth under 500 is greatly reduced. The per-base depth profiles, obtained by counting the number of collapsed reads aligning to each base in an approximately 56 kB region of interest, also shows a narrower distribution (FIG. 6A, FIG. 6B).

### Example 2: Balanced Capture Probe Library with Minimum Sequencing Depth

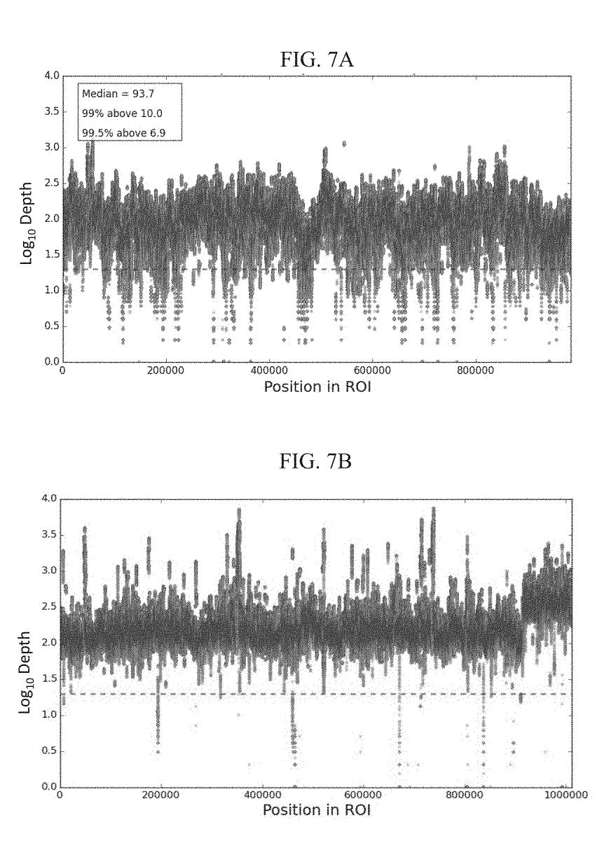

A capture probe library was constructed by combining >12,000 DNA capture probes at approximately equimolar amounts. The capture probes were 40 bases in length and are substantially complementary to various portions of a region of interest approximately 1,000,000 bases in length (in non-contiguous sub-regions). A DNA sequence complementary to a DNA oligonucleotide bound to the surface of an Illumina sequencing flow cell was fused to the 3′ end of the capture probe. The oligonucleotides bound to the surface of the Illumina sequencing flow cell were extended using the capture probe as template, thereby resulting in an Illumina sequencing flow cell with capture probes bound to the flow cell. Ninety-six sequencing libraries were then passed over the flow cell, thereby allowing nucleic acid molecules comprising substantially complementary segments to hybridize to the capture probes bound to the flow cell. The capture probes were extended using the nucleic acid molecules as a template, bridge amplified, and sequenced using an Illumina HiSeq2500. The log₁₀ sequencing depth attributable to the second percentile of each capture probe (that is, the sequencing depth reported is the second lowest sequencing depth for each capture probe among the 96 samples) in the capture probe library plotted against the position within the region of interest is shown in FIG. 7A. Six percent of the region of interest had a sequencing depth less than 20 (the predetermined minimum sequencing depth). The median sequencing depth among the approximately 1,000,000 loci in the region of interest is 93.7 (non-log), with 99% of loci having a sequencing depth above 10.0 (non-long) and 99.5% of loci having a sequencing depth above 6.9 (non-log).

For each probe in the capture probe library, a sequencing depth profile was constructed. The sequencing depth was plotted against the distance from the capture probe, and a median and interquartile range was determined at each 5-base locus for up to 500 bases (100 loci) from the capture probe. After 100 loci, the sequencing depth was assumed to be zero. A profile was constructed for each capture probe by subtracting two times the interquartile range from the median at each base. Probes that performed lower than the 1st percentile or higher than the 99th percentile, or fell outside of four-times the interquartile range, in terms of sequencing depth at the initial base or in terms of rate of decay were removed. The region of interest was divided into >5,000 tiles, and the capture probes were assigned to the nearest single tile or to a tile that the probe was specifically designed to cover. For each tile, an amount of each capture probe simulated from one of eight different amounts ranging from 0.2-fold to 8-fold of the initial amount, and a simulated sequencing depth for each capture probe was determined based on the simulated capture probe amount and the determined sequencing depth. For each of the one or more contiguous loci within the tile, the sum of the simulated sequencing depths of the capture probes was compared to a minimum sequencing depth of 20 for each combination of simulated capture probe amounts. The combination of the simulated amounts of capture probes that yielded the lowest simulated sequencing depth greater than the minimum sequencing depth of 20 was selected. Simulated capture probe amounts and sequencing depths were re-determined using a narrower window of capture probe amounts for a total of three iterations, with the final simulated capture probe amounts being used as the subsequent capture probe amount. A balanced capture probe library was constructed using the subsequent capture probe amounts and was used to enrich the 96 sequencing libraries. The enriched sequencing libraries were sequenced, and the log₁₀ sequencing depth attributable to the second percentile of each capture probe in the capture probe library plotted against the position within the region of interest is shown in FIG. 7B. Less than 1% of the region of interest had a sequencing depth less than 20 (the predetermined minimum sequencing depth).

1. A method of preparing a balanced capture probe library comprising:
sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library;
determining a sequencing depth attributable to each capture probe;
selecting a subsequent known amount of each capture probe based on the initial known amount and the sequencing depth attributable to said capture probe, wherein the subsequent known amount of each capture probe is selected to minimize a difference between a predicted sequencing depth profile and a desired sequencing depth profile, and wherein minimization of the difference is constrained by a minimum subsequent amount or a maximum subsequent amount of each capture probe; and
constructing the balanced capture probe library by combining at least a fraction of the capture probes at the subsequent known amount of each capture probe in the balanced capture probe library.

2. The method of claim 1, wherein the desired sequencing depth profile is non-uniform.

3. A method of preparing a balanced capture probe library comprising:
sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library;
determining a sequencing depth attributable to each capture probe;
selecting a subsequent known amount of each capture probe based on the initial known amount and the sequencing depth attributable to said capture probe, wherein the subsequent known amount of each capture probe is selected to minimize a difference between a predicted sequencing depth profile and a non-uniform desired sequencing depth profile; and
constructing the balanced capture probe library by combining at least a fraction of the capture probes at the subsequent known amount for each capture probe in the balanced capture probe library.

4. The method of claim 1, wherein the difference between the predicted sequencing depth profile and the desired sequencing depth profile is defined by an objective function.

5. The method of claim 4, wherein the desired sequencing depth profile is implied by the objective function.

6. The method of claim 1, wherein the initial known amount is an initial known volume and the subsequent known amount is a subsequent known volume.

7. The method of claim 1, wherein the initial known amount is an initial known mass or an initial known number of moles, and the subsequent known amount is a subsequent known mass or a subsequent known number of moles.

8. The method of claim 1, wherein the initial known amount is an initial known relative amount and the subsequent known amount is a subsequent known relative amount.

9. The method of claim 1, wherein the sequencing depth is based on a number of sequencing reads that comprise a sequence substantially complementary to a sequence within the capture probe.

10. The method of claim 1, wherein the sequencing depth is based on a number of sequencing reads that comprise a sequence substantially complementary to at least half of the capture probe.

11. The method of claim 9, wherein the number of sequencing reads is a number of consensus sequencing reads.

12. The method of claim 9, wherein the number of sequencing reads is a number of duplex consensuses sequencing reads.

13. The method of claim 1, wherein the capture probes are not substantially complementary to overlapping portions of the region of interest.

14. The method of claim 1, wherein at least two capture probes in the plurality of capture probes are substantially complementary to overlapping portions of the region of interest.

15. The method of claim 1, wherein the method comprises obtaining for each capture probe a binding fraction based on the sequencing depth attributable to the capture probe; and wherein the subsequent known amount is based on the initial known amount and the binding fraction.

16. The method of claim 1, wherein the method comprises obtaining for each capture probe a binding fraction, wherein the binding fraction is determined for the fraction of nucleic acid molecules comprising a segment substantially complementary to at least half of the capture probe that bound to the capture probe during enrichment of the sequencing library based on the sequencing depth attributable to the capture probe; and wherein the subsequent known amount is based on the initial known amount and the fraction.

17-19. (canceled)

20. The method of claim 1, wherein the difference is an objective function defined by a coefficient of variation.

21. The method of claim 1, wherein the plurality of capture probes comprises 10 or more unique capture probes.

22. The method of claim 1, wherein the capture probes are about 20 to about 160 bases in length.

23. The method of claim 1, wherein the plurality of capture probes comprise DNA capture probes.

24. The method of claim 1, wherein the plurality of capture probes comprise RNA capture probes.

25. The method of claim 1, wherein the capture probes are biotinylated.

26. The method of claim 1, further comprising:
enriching the sequencing library using the balanced capture probe library;
sequencing the sequencing library enriched using the balanced capture probe library;
determining a sequencing depth attributable to each capture probe in the balanced capture probe library;
selecting a second subsequent known amount of each capture probe based on the subsequent known amount of said capture probe in the balanced capture probe library and the sequencing depth attributable to said capture probe, wherein the second subsequent known amount of each capture probe is selected to minimize a difference between a second predicted sequencing depth profile and the desired sequencing depth profile; and
constructing a re-balanced capture probe library by combining at least a fraction of the capture probes at the second subsequent known amount of each capture probe in the re-balanced capture probe library.

27. The method of claim 26, wherein minimization of the difference is constrained by a minimum second subsequent amount or a maximum second subsequent amount of each capture probe.

28. The method of claim 1, wherein constructing the balanced capture probe library comprises combining each capture probe in the first capture probe library at the subsequent known amount of each capture probe in the balanced capture probe library.

29. The method of claim 1, wherein the sequencing library is an RNA sequencing library or a DNA sequencing library.

30. A balanced capture probe library made according to the method of claim 1.

31. A method of enriching a test sequencing library comprising:
combining the test sequencing library comprising nucleic acid molecules with the capture probe library prepared according to the method of claim 1; and
selecting nucleic acid molecules from the test sequencing library that hybridize with the capture probes in the capture probe library.

32. A method of sequencing a test sequencing library comprising:
enriching the test sequencing library according to the method of claim 31; and
sequencing the enriched test sequencing library.

33. The method of claim 31, further comprising amplifying the enriched test sequencing library.

34. The method of claim 1, further comprising removing the capture probes from the enriched test sequencing library.

35. The method of claim 31, wherein the test sequencing library comprises cell-free DNA.

36. The method of claim 35, wherein the cell-free DNA comprises fetal cell-free DNA.

37. The method of claim 35, wherein the cell-free DNA comprises circulating tumor cell-free DNA.

38. The method of claim 31, wherein the test sequencing library comprises fragmented DNA derived from cells contained with a sample.

39. The method of claim 31, wherein the test sequencing library is an RNA sequencing library.

40. The method of claim 31, wherein the nucleic acid molecules in the test sequencing library have an average length of about 100 bases to about 500 bases.

41. The method of claim 31, wherein the nucleic acid molecules in the test sequencing library are ligated to sequencing adapters comprising molecular barcodes.

42. The method of claim 1, wherein enriching the sequencing library comprises separating nucleic acid molecules in the sequencing library that are hybridized to the capture probes from nucleic acid molecules in the sequencing library that are not hybridized to the capture probes.

43. A method of forming a pooled capture probe library comprising combing two or more balanced capture probe libraries prepared according to the method of claim 1.

44. A method of preparing a balanced capture probe library comprising:
sequencing a sequencing library comprising a plurality of nucleic acid molecules enriched using a first capture probe library comprising a plurality of capture probes, wherein each capture probe comprises a sequence that is substantially complementary to a portion of or adjacent to a region of interest included in the sequencing library, and wherein an initial known amount of each capture probe is used to form the first capture probe library;
determining at each locus within a tile of the region of interest comprising a one or more contiguous loci:
i. a sequencing depth attributable to each capture probe in the first capture probe library, and
ii. a sequencing depth attributable to the first capture probe library;
selecting a subsequent known amount of each capture probe based on the initial known amount of said capture probe, the sequencing depth at each locus attributable to said capture probe, and the sequencing depth at each locus attributable to the first capture probe library, wherein the subsequent amount is selected to obtain a desired sequencing depth profile at substantially all of the one or more contiguous loci within the tile; and
constructing the balanced capture probe library by combining at least a fraction of the capture probes at the subsequent known amount of each capture probe in the balanced capture probe library.

45-73. (canceled)
